(19) United States
(12) Patent Application Publication
(10) Pub. No.: US 2024/0047012 A1
(43) Pub. Date: Feb. 8, 2024

(54) METHODS AND SYSTEMS FOR PREDICTING FUNCTION BASED ON RELATED BIOPHYSICAL ATTRIBUTES IN DATA MODELING

(71) Applicant: GENENTECH, INC., South San Francisco, CA (US)

(72) Inventors: Alexander KOZINTSEV, San Jose, CA (US); Tilman Sebastian SCHLOTHAUER, South San Francisco, CA (US); Raul Agustin SUN HAN CHANG, South San Francisco, CA (US)

(73) Assignee: GENENTECH, INC., South San Francisco, CA (US)

(21) Appl. No.: 18/452,296

(22) Filed: Aug. 18, 2023

Related U.S. Application Data
(63) Continuation of application No. PCT/US22/16157, filed on Feb. 11, 2022.
(60) Provisional application No. 63/151,527, filed on Feb. 19, 2021.

(51) Int. Cl. G16B 40/20; G16B 5/00

(52) U.S. Cl. CPC G16B 40/20; G16B 5/00

(57) ABSTRACT

Methods and systems may be provided to predict functional response based on a set of predictors for therapeutic proteins. For example, a method can comprise receiving input data comprising first input data related to a set of predictors and corresponding measured functional response associated with the set of predictors obtained from a first set of therapeutic protein samples and second input data related to the set of predictors and a second set of therapeutic protein samples for prediction of a functional response, wherein the set of predictors were selected as a combination of related biophysical attributes of therapeutic proteins based on a pre-determined criterion; training a machine learning model with the first input data; and using the machine learning model and the set of predictors to predict a functional response of the second set of therapeutic protein samples based on the second input data.

### CROSS-REFERENCE

The application is a Continuation application under the benefit of 35 U.S.C. 365(c) of International Application No. PCT/US2022/016157, entitled “Methods and Systems for Predicting Function Based on Related Biophysical Attributes in Data Modeling,” filed Feb. 11, 2022, which claims priority to U.S. Provisional Patent Application No. 63/151,527, entitled “Methods and Systems for Predicting Function Based on Related Biophysical Attributes in Data Modeling,” filed Feb. 19, 2021, which are incorporated herein by reference in their entirety.

### FIELD

Provided herein are methods and systems for improved prediction of function response of proteins such as antibodies. More specifically, methods and systems are provided for using multiple biophysical attributes to predict related function response of antibodies.

### BACKGROUND

Prior data modeling approaches for correlating biophysical attributes to functional assays have relied on a linear relationship between a single biophysical attribute and function using data from only one single biophysical attribute. This prior approach often neglects the contributing impacts of multiple other biophysical attributes that have also been shown or that may potentially modulate the function of interest and is laborious to use in the investigation of the interaction effects between the biophysical attributes themselves. There remains a need for developing improved ways to more accurately predict functional response using multiple predictors such as biophysical attributes.

### SUMMARY

Methods and systems may be provided to predict functional response based on a set of predictors for therapeutic proteins. For example, a method can comprise receiving input data comprising first input data related to a set of predictors and corresponding measured functional response associated with the set of predictors obtained from a first set of therapeutic protein samples and second input data related to the set of predictors and a second set of therapeutic protein samples for prediction of a functional response, wherein the set of predictors were selected as a combination of related biophysical attributes of therapeutic proteins based on a pre-determined criterion; training a machine learning model with the first input data. The method can further comprise using the machine learning model and the set of predictors to predict a functional response of the second set of therapeutic protein samples based on the second input data; and returning an output comprising the predicted functional response. For example, the therapeutic protein samples can be antibody samples, the functional response can be antibody-dependent cell-mediated cytotoxicity (ADCC) response, complement-dependent cytotoxicity (CDC) response. Fc gamma receptors (FcyR) binding or complement C1q binding, and the related biophysical attributes of therapeutic proteins comprise a degree of afucosylation and one or more additional glycosylation attributes of antibodies.

In various embodiments, a system can comprise a data source for obtaining one or more datasets, wherein the one or more datasets comprise: a) first input data related to a set of predictors and corresponding measured functional response associated with the set of predictors obtained from a first set of therapeutic protein samples and b) second input data related to the set of predictors and a second set of therapeutic protein samples for prediction of a functional response, wherein the set of predictors were selected as a combination of related biophysical attributes of therapeutic proteins based on a pre-determined criterion; a computing device communicatively connected to the data source and configured to receive the dataset, the computing device comprising a non-transitory computer readable storage medium containing instructions which, when executed on one or more data processors, cause the one or more data processors to perform a method, the method comprising: training a machine learning model with the first input data; using the machine learning model and the set of predictors to predict a functional response of the second set of therapeutic protein samples based on the second input data; and returning an output comprising the predicted functional response.

In various embodiments, there can be provided a computer-program product tangibly embodied in a non-transitory machine-readable storage medium, including instructions configured to cause one or more data processors to perform a method for selecting a cell of interest based on a single cell dataset, the method comprising: receiving input data comprising: a) first input data related to a set of predictors and corresponding measured functional response associated with the set of predictors obtained from a first set of therapeutic protein samples and b) second input data related to the set of predictors and a second set of therapeutic protein samples for prediction of a functional response, wherein the set of predictors were selected as a combination of related biophysical attributes of therapeutic proteins based on a pre-determined criterion; training a machine learning model with the first input data; using the machine learning model and the set of predictors to predict a functional response of the second set of therapeutic protein samples based on the second input data; returning an output comprising the predicted functional response.

The terms and expressions which have been employed arc used as terms of description and not of limitation, and there is no intention in the use of such terms and expressions of excluding any equivalents of the features shown and described or portions thereof, but it is recognized that various modifications are possible within the scope of the claimed embodiments. Thus, it should be understood that although the present claimed embodiments have been specifically disclosed as embodiments and optional features, modification and variation of the concepts herein disclosed may be resorted to by those skilled in the art, and that such modifications and variations are considered to be within the scope of the appended claims.

### BRIEF DESCRIPTION OF THE DRAWINGS

The present disclosure is described in conjunction with the appended figures:

FIG. 1 illustrates non-limiting exemplary embodiments of a general schematic workflow for predicting functional activity based on a selected combination of related biophysical attributes, in accordance with various embodiments.

FIG. 2 illustrate a non-limiting example process for developing a model for using multiple biophysical attributes to predict related functional response, in accordance with various embodiments.

FIG. 3 illustrates non-limiting exemplary embodiments of a general schematic workflow 300 for predicting functional activity based on a selected combination of related biophysical attributes, in accordance with various embodiments.

FIG. 4A illustrates non-limiting exemplary embodiments of a graph showing a correlation plot of all the variables compared.

FIG. 4B illustrates non-limiting exemplary embodiments of a graph showing variations within the samples and further determine correlations between the predictors.

FIG. 5 illustrates non-limiting exemplary embodiments of a graph showing a ranking of the predictors by calculating the relative contribution of each predictor to the model for variable importance ranking.

FIG. 6 illustrates non-limiting exemplary embodiments of a graph showing results from a feature selection method. This feature selection method runs every possible combination of predictors through the computationally taxing and more rigorous repeated random subsampling validation.

FIG. 7 illustrates non-limiting exemplary embodiments of a graph showing results from a feature selection method. This feature selection method runs only a group of top performing predictor subsets from a preliminary moderate validation through repeated random subsampling validation.

FIGS. 8A-8B illustrates non-limiting exemplary embodiments of a graph showing model performance validation in residual analysis (FIG. 8A) and recovery analysis (FIG. 8B).

FIG. 9 is a flowchart illustrating a method for predicting functional activity based on related biophysical attributes, in accordance with various embodiments.

FIG. 10 illustrates non-limiting exemplary embodiments of a system for predicting functional activity based on related biophysical attributes, in accordance with various embodiments.

FIG. 11 is a block diagram of non-limiting examples illustrating a computer system configure to perform methods provided herein, in accordance with various embodiments.

### DETAILED DESCRIPTION

### I. Overview

The application of machine learning to the modelling of structure-function relationships helps to address a difficult challenge unique to the biological complexity of biotherapeutics, considering the compounded and synergistic effects of multiple biophysical attributes such as modified structural attributes on one biologically-relevant functional response. Biotherapeutics are susceptible to different structural modifications throughout production and subsequent processing, leading to distributions of individual modified structural attributes being present downstream in the population of molecules comprising a manufactured lot. In order to ensure the quality of a biotherapeutic, manufacturing process control strives to ensure the reproducible production of biotherapeutic lots with similar distributions of critical modifications. However, in order to set appropriate limits on the acceptable levels of modifications, scientists must first demonstrate that within a certain range (or below a certain limit) of a modification or impurity, a biotherapeutic product will maintain a safe and efficacious functional profile.

Scientists accomplish this goal in several ways: leveraging studies from animal models, researching creditable prior knowledge, referencing clinical exposure levels, and by correlating levels of critical modifications with biologically-relevant in vitro functional characterization. Due to there being different distributions of modifications present in a single lot but low diversity of those distributions between manufacturing lots, meaningful quantitative relationships of the different individual modified structural attributes with a biologically-relevant function are difficult to deconvolute. This is further complicated by the fact most biologically-relevant in vitro functions are significantly impacted by multiple structural attributes, working collaboratively in either an additive or synergistic manner. Although scientists can generate or isolate some modified structural variants, in doing so they only facilitate the modelling of univariate structure-function impacts which, when combined, would still be unable to incorporate synergistic effects of different structural modifications.

As described herein, a uniquely-suited solution to this biological and analytical problem is provided by the use of machine learning modelling, which reduces the complexity coming from biological modification dimensionality and elicits relevant quantitative relationships based on the holistic structural characterization profile of a biotherapeutic.

For example, during clinical and commercial manufacture of therapeutic antibodies, such as monoclonal human antibodies (mAbs), the biophysical and functional characteristics of the therapeutic antibodies can be carefully monitored in order to ensure process and quality control. This data collected in monitoring can be leveraged to use individual structural attributes to predict biologically-relevant functional responses and therefore to guide the calculation of acceptance criteria for release. In cases where one structural attribute of the therapeutic antibodies has a profoundly large effect on a particular functional response of the therapeutic antibodies, such univariate correlations can serve as powerful predictive models; however, in cases where multiple structural attributes impact a biologically-relevant functional response on a similar scale, univariate correlations between a single structural attribute and the related functional response are less useful.

Methods and systems described herein can leverage multiple predictors such as multiple biophysical attributes (e.g., structural attributes) for larger sets of data from individual molecules and from sets of multiple molecules of a similar class (e.g. antibodies such as CHO-derived IgG1 therapeutics) to generate robust linear and non-linear models. In various embodiments, methods and systems described herein can simultaneously perform principal component analyses to visualize and approximately quantify the relationships between the predictors with the response and with each other and can therefore identify and select relevant predictors for predicting the functional response based on the relationships.

In various embodiments, methods and systems described herein can be applied for predicting a functional response of therapeutic proteins, such as in vitro antibody-dependent cellular cytotoxicity (ADCC) response of antibodies. For example, the correlation of in vitro ADCC and the level of afucosylated glycan species and one or more other biophysical attributes of antibodies or fragments thereof can be used to predict ADCC response and therefore therapeutic efficacy of the antibodies or fragments thereof.

Non-limiting biophysical attributes of proteins such as therapeutic glycoproteins (e.g., antibodies) can include, but not be limited to, Fc N-glycan structures, glycan species of Fc regions (such as highly galactosylated forms, forms of high mannose), the degree of overall glycosylation of Fc regions, and the presence of certain post-translational modifications in the Fc. Methods and systems described herein can be used to predict a functional response such as ADCC response based on multiple biophysical attributes like afucosylated glycan species or other glycan species of Fc regions, the degree of overall glycosylation of Fc regions, and the presence of certain post-translational modification on the Fc, or any combination thereof.

In accordance with various embodiments, the therapeutic proteins or antibodies can include multi-valent IgG-like molecules, such as bispecifics, or engineered Fab fragments, such as dual-targeting engineered Fab fragments that can bind two antigens.

In various embodiments, the therapeutic proteins or antibodies' functional response can include, for example, antibody-dependent cell-mediated cytotoxicity (ADCC) response, complement-dependent cytotoxicity (CDC) response, Fc gamma receptors (FcyR) binding or complement Clq binding, and the related biophysical attributes of therapeutic proteins or antibodies including, for example, glycosylation attributes, deamidation in the Fc (VSNK), low or high molecular weight forms. For example, the glycosylation attributes can include a degree of afucosylation, galactosylation, sialylation, glycan chain length, glycan building block type, and forms of antibodies missing N-glycan chains, or any combination thereof.

In accordance with various embodiments, the therapeutic proteins or antibodies' functional response can include, for example, pharmacokinetic clearance or neonatal Fc receptor (FcRn) binding, and the related biophysical attributes of therapeutic proteins or antibodies can include, for example, site-specific modifications in Fc or charged variants of Fab.

In accordance with various embodiments, the therapeutic proteins or antibodies' functional response can include, for example, cell-based immuno potency or activity and target binding, and the related biophysical attributes of therapeutic proteins or antibodies can include, for example, site-specific modifications in CDR, charge and size variants, disulfide mispairing and free thiols.

In accordance with various embodiments, the therapeutic proteins or antibodies' functional response can include, for example, immunogenicity, and the related biophysical attributes of therapeutic proteins or antibodies can include, for example, clipping, size forms, or mispairing of light chain or half antibody in bispecific antibodies.

For example, in cases in which large amounts of biophysical and functional characterization data are already available, such as in late stage technical development of biotherapeutics, such methods and systems allow for an enhancement of product knowledge, and can contribute to the setting of specifications for manufacturing control and even identifying and selecting therapeutic candidates for therapeutic development.

This disclosure describes various exemplary embodiments for using multiple biophysical attributes to predict related functional response, such as, for example, an ADCC response of therapeutic proteins such as, for example, antibodies. The disclosure, however, is not limited to these exemplary embodiments and applications or to the manner in which the exemplary embodiments and applications operate or arc described herein. Moreover, the figures may show simplified or partial views, and the dimensions of elements in the figures may be exaggerated or otherwise not in proportion.

### II. Definitions

It is to be understood that the terminology used herein is for the purpose of describing particular embodiments only, and is not intended to be limiting.

Unless defined otherwise, all terms of art, notations and other technical and scientific terms or terminology used herein arc intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Generally, nomenclatures utilized in connection with, and techniques of, chemistry, biochemistry, molecular biology, pharmacology and toxicology are described herein are those well-known and commonly used in the art.

As used herein, the singular forms “a” “an” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes” “including” “comprises” and/or “comprising” when used herein, specify the presence of stated features, integers, steps, operations, elements, components, and/or units but do not preclude the presence or addition of one or more other features, integers, steps, operations, elements, components, units, and/or groups thereof.

Throughout this disclosure, various aspects are presented in a range format. It should be understood that the description in range format is merely for convenience and brevity and should not be construed as an inflexible limitation on the disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed in the disclosure. The upper and lower limits of these smaller ranges may independently be included in the smaller ranges, and arc also encompassed in the disclosure, subject to any specifically excluded limit in the stated range. Where the stated range includes one or both of the limits, ranges excluding either or both of those included limits are also included in the disclosure. This applies regardless of the breadth of the range.

As used herein, the term “antibody” is intended to refer broadly to any immunologic binding agent such as IgG, IgM, IgA, IgD and IgE as well as polypeptides comprising antibody CDR domains that retain antigen binding activity. Thus, the term “antibody” is used to refer to any antibody molecule that has an antigen binding region, and includes antibody fragments such as Fab′, Fab, F(ab′)2, single domain antibodies (DABs), Fv, scFv (single chain Fv), and polypeptides with antibody CDRs, scaffolding domains that display the CDRs (e.g., anticalins) or a nanobody.

As used herein, the term “Fc” or a crystallizable fragment refers to a fragment of an antibody that interacts with cell surface receptors called Fc receptors and some proteins of the complement system. Fc is relatively constant and encodes the isotype for a given antibody; this Fc region can also confer additional functional capacity through processes such as antibody-dependent complement deposition, cellular cytotoxicity, cellular trogocytosis, and cellular phagocytosis. The term “Fab”, also referred to as an antigen-binding fragment, refers to the variable portions of an antibody molecule with a paratope that enables the binding of a given epitope of a cognate antigen. The amino acid and nucleotide sequences of the Fab portion of antibody molecules are hypervariable.

As used herein, the term “antibody-dependent cellular cytotoxicity (ADCC),” also referred to as antibody-dependent cell-mediated cytotoxicity, is a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can act to limit and contain infection.

As used herein, the term “biophysical attribute” can refer to any values determined from a biophysical assay of a biological molecule, such as an antibody molecule (including fragments thereof). For example, the biophysical attribute of a glycoprotein such as an antibody molecule can include any post-translational modification, glycan structure, or charge and size species, afucosylated glycan species or other glycan species (e.g., galactosylated glycan species, mannose form, sialylated species, etc.), the degree of overall glycosylation, and the presence of certain post-translational modification, or any combination thereof. The biophysical attribute of an antibody molecule can be a modification or structure of particular region, such as an Fc region of the antibody molecule, like afucosylated glycan species or other glycan species of an Fc region.

A fucosylated form of a protein, as used herein, refers to a glycan structure having at least a fucose moiety. An afucosylated form of a protein, as used herein, refers to a glycan structure lacking a fucose moiety. A galactosylated form of a protein, as used herein, refers to a glycan structure having at least a galactose monosaccharide moiety. A mannose form of a protein, as used herein, refers to a glycan structure having at least a mannose moiety. A sialylated form of a protein, as used herein, refers to a glycan structure having at least a sialylated moiety.

As used herein, “glycan” refers to a sugar, which can be monomers or polymers of sugar residues, such as at least three sugars, and can be linear or branched. A “glycan” can include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g., of a glycoprotein, glycolipid, proteoglycan, etc.). The term also encompasses free glycans, including glycans that have been cleaved or otherwise released from a glycoconjugate.

As used herein, the term “glycoprotein” refers to a protein that contains a peptide backbone covalently linked to one or more sugar moieties (i.e., glycans), such as an antibody. The sugar moieties may be in the form of monosaccharides, disaccharides, oligosaccharides, and/or polysaccharides. The sugar moieties may comprise a single unbranched chain of sugar residues or may comprise one or more branched chains. Glycoproteins can contain O-linked sugar moieties and/or N-linked sugar moieties.

The term “CDR (Complementarity-Determining Region),” as used herein, refers to complementarity-determining regions that are the portions of the amino acid sequence of a T or B cell receptor and are predicted to bind to an antigen.

The term “about”, as used herein, refers to include the usual error range for the respective value readily known. Reference to “about” a value or parameter herein includes (and describes) embodiments that are directed to that value or parameter per se. For example, description referring to “about X” includes description of “X”. In various embodiments, “about” may refer to ±15%, ±10%, ±5%, or ±1% as understood by a person of skill in the art.

In addition, as the terms “coupled with” or “communicatively coupled with” or similar words are used herein, one element may be capable of communicating directly, indirectly, or both with another element via one or more wired communications links, one or more wireless communications links, one or more optical communications links, or a combination thereof. In addition, where reference is made to a list of elements (e.g., elements a, b, c), such reference is intended to include any one of the listed elements by itself, any combination of less than all of the listed elements, and/or a combination of all of the listed elements.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “ones” means more than one.

As used herein, the term “plurality” or “group” can be 2, 3, 4, 5, 6, 7, 8, 9, 10, or more.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” or “at least one of item A, item B, and item C” may mean item A; item A and item B; item B; item A, item B, and item C; item B and item C; or item A and C. In some cases, “at least one of item A, item B, or item C” or “at least one of item A, item B, and item C” may mean, but is not limited to, two of item A, one of item B, and ten of item C; four of item B and seven of item C; or some other suitable combination.

An “individual”, “subject,” or “patient” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

The headers and subheaders between sections and subsections of this document are included solely for the purpose of improving readability and do not imply that features cannot be combined across sections and subsection. Accordingly, sections and subsections do not describe separate embodiments.

Various embodiments of the present disclosure include a system including one or more data processors. In various embodiments, the system includes a non-transitory computer readable storage medium containing instructions which, when executed on the one or more data processors, cause the one or more data processors to perform part or all of one or more methods and/or part or all of one or more processes disclosed herein. Various embodiments of the present disclosure include a computer-program product tangibly embodied in a non-transitory machine-readable storage medium, including instructions configured to cause one or more data processors to perform part or all of one or more methods and/or part or all of one or more processes disclosed herein.

This description provides exemplary embodiments only, and is not intended to limit the scope, applicability or configuration of the disclosure. Rather, the ensuing description of exemplary embodiments will provide those skilled in the art with an enabling description for implementing various embodiments. It is understood that various changes may be made in the function and arrangement of elements without departing from the spirit and scope as set forth in the appended claims.

Specific details arc given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In various instances, well-known circuits, processes, algorithms, structures, and techniques may be shown without unnecessary detail in order to avoid obscuring the embodiments.

All references cited herein, including patent applications, patent publications, and UniProtKB/Swiss-Prot Accession numbers are herein incorporated by reference in their entirety, as if each individual reference were specifically and individually indicated to be incorporated by reference.

### III. Prediction of Functional Activity Based on Biophysical Attributes

Various method and system embodiments described herein enable using multiple biophysical attributes to predict related functional response, such as an ADCC response or binding to a desired target, e.g., a desired antigen. For example, the methods and systems described herein may be used to leverage one or more statistical models and machine learning models to identify correlations between biophysical attributes and functional characterization data and build predictive models that take as input measured biophysical attributes and outputs predicted functional characterization. The embodiments described herein can be sensitive and reproducible and can enable more accurate prediction of the functional response.

III.A. Workflows

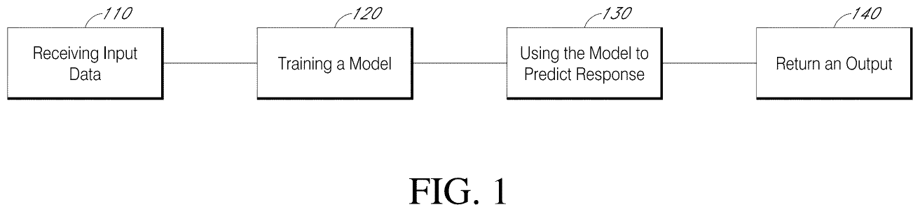

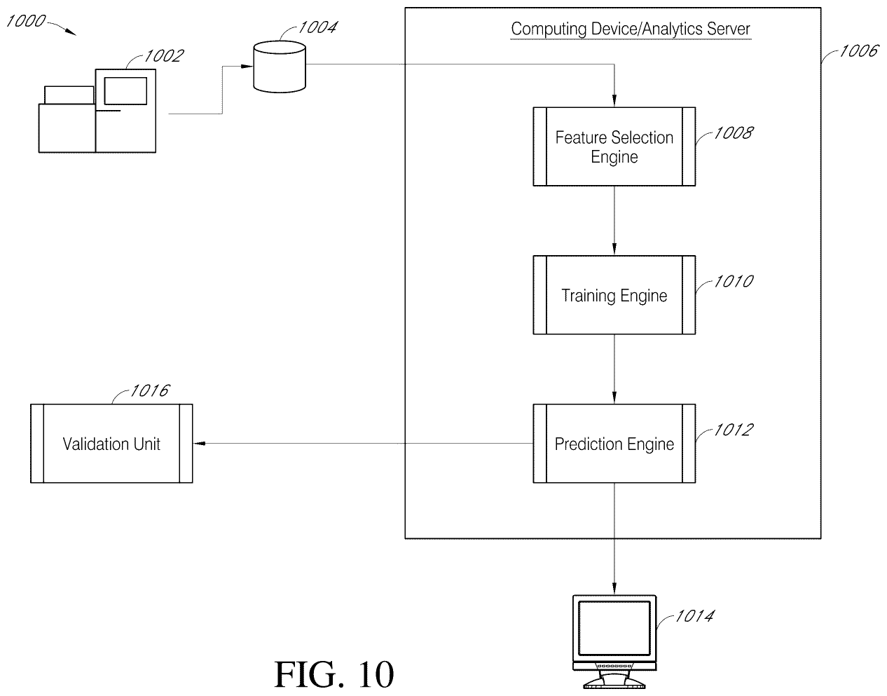

FIG. 1 illustrates non-limiting exemplary embodiments of a general schematic workflow for predicting functional activity based on a selected combination of related biophysical attributes, in accordance with various embodiments. The workflow 100 can include various combinations of features, whether it be more or less features than that illustrated in FIG. 1. As such, FIG. 1 simply illustrates one example of a possible workflow. The workflow 100 may be implemented using, for example, system 1000 described with respect to FIG. 10 or a similar system.

In various embodiments, the workflow 100 can be automated. The workflow 100 can include, at step 110, receiving input data. The input data can include first input data related to a set of predictors (e.g., biophysical attributes) and corresponding functional response (e.g., measured antibody-dependent cellular cytotoxicity (ADCC) response) associated with the set of predictors obtained from a first set of therapeutic protein (e.g., antibody) samples. The first input data can include labeled data with a correlation between the biophysical attribute data and functional data of the same set of samples for training a model.

The input data can further include second input data related to a second set of therapeutic protein (e.g., antibody) samples for prediction of a function response using the model trained by the first input data. The second input data can be unlabeled data and can include biophysical attribute data for prediction of a function response such as ADCC response.

Biophysical attribute data, also referred to as “predictor” data, can be obtained from research and development, process validation, or GMP testing, and can come from multiple physical assays such as, for example, Labeled Released Glycan hydrophilic interaction liquid chromatography (HILIC) analysis, Non-Reduced and Reduced Capillary electrophoresis sodium dodecyl sulfate (CE-SDS), Ion Exchange Chromatography, Size Exclusion Chromatography, and imaged capillary iso-electric focusing (iCIEF).

Functional data, also referred to and “response” data, can also be obtained from research and development, process validation, or GMP testing and can come from multiple molecule-specific or platform cell-based in vitro activity assays.

The workflow 100 can include, at step 120, training a model with the first input data. The first input data, e.g., the labeled data comprising the selected subsets of predictors (selected from predictors including, but not limited to, glycans, charge and size species, peptide modifications) and the functional response of interest (including, but not limited to, potency, receptor binding, ADCC response), can be first inputted into the workflow 100 for training a model.

The model can be a user-selected model or an automatically-selected model, such as a regression and classification statistical model or machine learning model. Non-limiting examples of the model can include a model based on partial least square, random forest, support vector machine, Naive Bayes, k-nearest neighbors (KNN), generalized additive model, logistic regression, gradient boosting, lasso, or any combination or modification thereof. The selection of an appropriate model can be a shotgun approach of one or more of the following steps, including categorizing statistical models and machine learning models into groups based on their best-case use (e.g., small or large sample size, strong non-linear behavior, etc.), analyzing the parameters of the dataset (e.g., sample size, linear vs. non-linear behavior, etc.), selecting the group of models that best fits the criteria of the dataset, and/or comparing the performance of all the models in this group at the feature selection step.

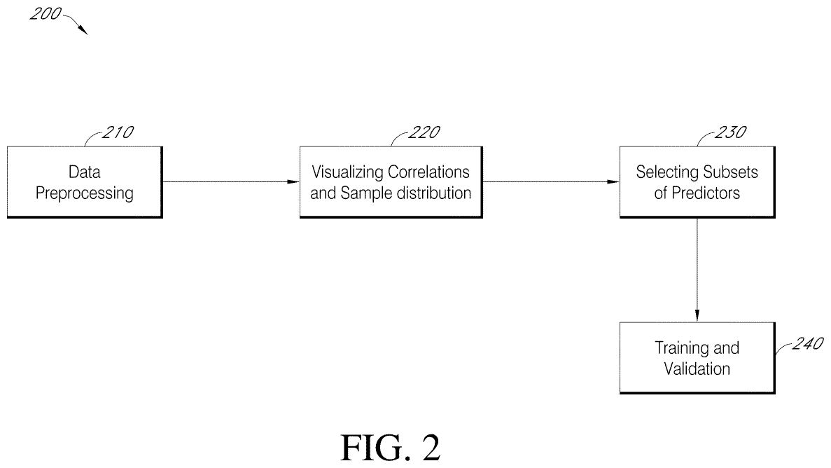

The training step 120 can include one or more steps as detailed in FIG. 2, such as, for example, visualizing correlations of some or all variables, determining sample distribution, identifying subsets of predictor used for training, training the model with data associated with identified subsets of predictors, and validating the model. Note that while FIG. 2 illustrate a series of connected steps, each and every step illustrated in FIG. 2 need not be present in executing training step 120.

The workflow 100 can include, at step 130, using the trained model to predict a functional response for samples that with unknown or undetermined functional response based on the first input data and second input data. The second input data relate to a second set of therapeutic protein (e.g., antibody) samples and can be inputted into the model trained by the first input data for prediction of the functional response of the second set of therapeutic protein (e.g., antibody) samples. For example, the first input data include a cleaned dataset comprising data based on a selected subset of predictors from feature selection and the response, wherein the data relate to samples with known values of predictors and response. The second input data relate to desired samples for prediction that contain measured values for the selected subset of predictors (no response values required, as these will be predicted by the fully trained model). The output of the prediction at step 130 can be predicted values for the functional response of the desired samples for prediction in the second input data.

The workflow 100 can include, at step 140. returning an output based on the predicted functional response. The output can be used to select antibody therapeutic candidates with a predicted functional response meeting a pre-defined criterion. The candidates can be validated by experiments to confirm their functional response and therapeutic value and be used for therapeutic development.

In accordance with various embodiments, a general and example schematic workflow 200 is provided in FIG. 2 to illustrate a non-limiting example process for developing a model for using multiple biophysical attributes to predict related functional response, such as an ADCC response. One or more steps of the workflow 200 can be incorporated into one or more step of the workflow 100 including, for example, the training step 120, in FIG. 1.

In various embodiments, the workflow 200 can be automated. The workflow 200 can include various combinations of features, whether it be more or less features than that illustrated in FIG. 2. As such, FIG. 2 simply illustrates one example of a possible workflow. The workflow 200 may be implemented using, for example, system 1000 described with respect to FIG. 10 or a similar system.

In various embodiments, the workflow 200 can include one or more of sequential data preprocessing, principal component analysis, feature selection, and training and validating a user-selected model such as a regression or classification statistical model or machine learning model, or a combination or a modification thereof.

The workflow 200 can include, at step 210, data preprocessing. Raw data including values for predictors and response can be received and cleaned in this step by omission or imputation of samples with missing values for predictors and response (e.g., samples with values for only predictors but not response or samples with values for only response but not predictors), especially for raw data related to a set of predictors and corresponding measured antibody-dependent cellular cytotoxicity (ADCC) response associated with the set of predictors obtained from a first set of therapeutic protein (e.g., antibody) samples.

The workflow 200 can include, at step 220, visualizing correlations between biophysical attributes and functional response and determining sample distribution using the cleaned data from the data preprocessing step 210. This step can be used to glean more information from the molecular datasets, for example, the sample distribution (possibility of identifying outliers) and collinearities between the predictors.

For example, a correlation plot analysis can be used to visualize the correlation between compared variables, including one or more predictors and functional response (e.g. sum of afucosylated in the Fc regions of antibodies, sum of galactosylated in the Fc regions of antibodies, sum of mannose in the Fc regions of antibodies, sum of sialylated in the Fc regions of antibodies, and ADCC) or combinations of the variables. The input for the correlation plot is the full cleaned dataset (omission or imputation of samples with missing values for predictors and response) containing all predictors and the desired response

For example, a principal component analysis (PCA) can be carried out to visualize variation within the samples and further determine correlations between any compared predictors or combinations thereof (e.g. sum of afucosylated in the Fc regions of antibodies, sum of galactosylated, sum of mannose in the Fc regions of antibodies, sum of sialylated in the Fc regions of antibodies). The input for the PCA can be, for example, a full cleaned dataset containing only predictors and no response.

The workflow 200 can include, at step 230, selecting subsets of predictors. The subset of predictors can include a combination of predictors that are predicted or determined to meet a pre-defined performance criterion, for example, the top first, second, third, fourth, fifth or any pre-defined top ranked combination of predictors. The subset of predictors may include a combination of at least or at most two, three, four, five, six, seven, nine, ten predictors. The subset of predictors can be selected from any biophysical attributes of antibodies or fragments thereof, such as amino acid integrity, the oligomeric state and the glycosylation pattern. In a various embodiments, the subset of predictors can be selected from any attributes of the glycosylation pattern, such as glycan species heterogeneity, the degree of overall glycosylation and the presence of certain post-translational modifications in the Fc region of antibodies or fragments thereof.

In various embodiments, every single possible combination of an initial set of predictors can undergo repeated random subsampling validation, whereby the data related to the initial set of predictors arc split into a train set used to build a model and a test set used to validate the model. The trained model predicts values for the test set sample responses, which are directly compared to the actual measured values to calculate the Root Mean Squared Error of Prediction (RMSEP) of that model. This is carried out through a user-defined number of iterations of random train and test set splits for every combination of the set of predictors. A subset of predictors can be selected for performance meeting a pre-defined criterion; for example, the subset of predictors yielding models with the best average predictive accuracy (lowest average RMSEP) is then automatically selected to move forward.

In accordance with various embodiments, the number of combinations of the initial set of predictors is initially reduced by running a preliminary k-fold cross-validation on every combination of the initial set of predictors. Rather than training and validating multiple models on different iterations of randomized train and test set splits, the data is split only once into a pre-defined k value of different groups, for example the pre-defined k value is five or ten or any value that is chosen so that each train/test group of data samples based on the k value is large enough to be statistically representative of the broader dataset. All the groups except for one are used as a training set to fit a model, which is then evaluated using the remaining groups as a test set. This process can be carried out until each group serves as a test set once, and the average performance for prediction of the test sets is reported. Similarly, a subset of predictors can be selected for performance meeting a pre-defined criterion based on the predicted performance.

In various embodiments, the input for step 230 is the full cleaned dataset containing all predictors and the desired response (e.g., full data for feature importance ranking and preliminary feature selection via 5-fold cross-validation, or train/test split data for full feature selection via repeated random subsampling). In various embodiments, the output for this step 230 is a ranked order of the relative contribution of each predictor to the model built for predicting the response and a subset of selected predictors to use for the model (e.g., data subset of predictors that trains the model with the best predictive performance for unseen samples).

The workflow 200 can include, at step 240, validation of the model performance. In various embodiments, the input for this step 240 is a cleaned dataset comprising, for example, data associated with the selected subset of predictors from feature selection at step 230 and the response corresponding to the selected subset of predictors followed by splitting into train/test split data. In various embodiments, the output for this step 240 is a statistically sound estimate (e.g., an empirical rule and a tolerance interval) for the range of error in the predictions of functional response for desired samples.

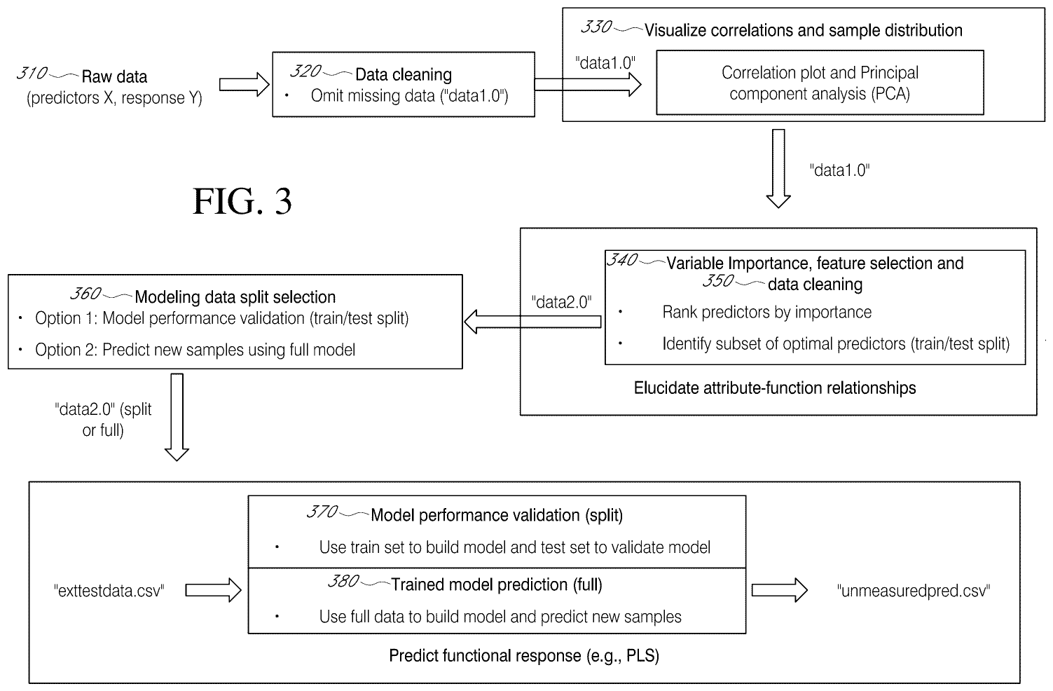

FIG. 3 illustrates non-limiting exemplary embodiments of a general schematic workflow 300 for predicting functional activity based on a selected combination of related biophysical attributes, in accordance with various embodiments. The workflow 300 can include various combinations of features, whether it be more or less features than that illustrated in FIG. 3. As such, FIG. 3 simply illustrates one example of a possible workflow. The workflow 300 may be implemented using, for example, system 1000 described with respect to FIG. 10 or a similar system.

In various embodiments, the workflow 300 can be automated. For example, the automated workflow 300 can be built using the programming language R, and can be run using any integrated development environment for R. In various embodiments, the predictive modeling is carried out using a software package, which can contain a set of functions that simplifies the process of creating predictive models for regression and classification problems.

In various embodiments, the workflow 300 utilizes a multivariate partial least square (PLS) regression model. This package can implement a kernel algorithm, which can be efficient when the number of predictors is larger than the number of samples. Further for example, PLS can be robust when predictors are highly collinear, which can be the case between correlated biophysical attributes.

For example, in order to investigate the impacts of multiple glycan attributes, hydrophilic interaction chromatography (HILIC) glycan data from across multiple CHO-derived IgG1 monoclonal antibodies (mAbs) (therapeutic mAb 1, 2, 3) was used to model in vitro ADCC functional response using the relative percent areas of glycan species obtained by 2-AB HILIC Glycan analysis. The modeling was done individually for each molecule, as well as in combination, in order to examine the translation of glycan structure impact on in vitro ADCC function response across the different molecules. The modeling was followed in an exemplary workflow as described in FIG. 3.

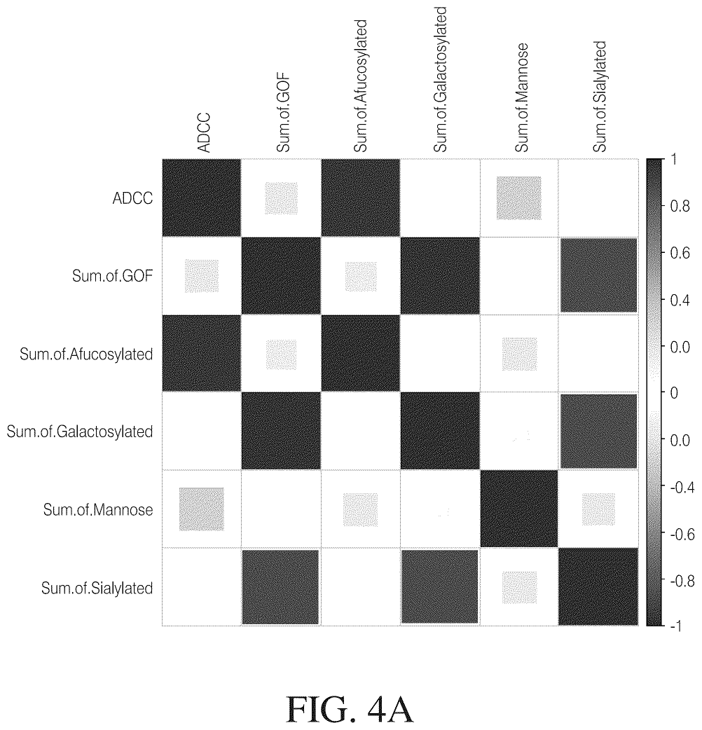

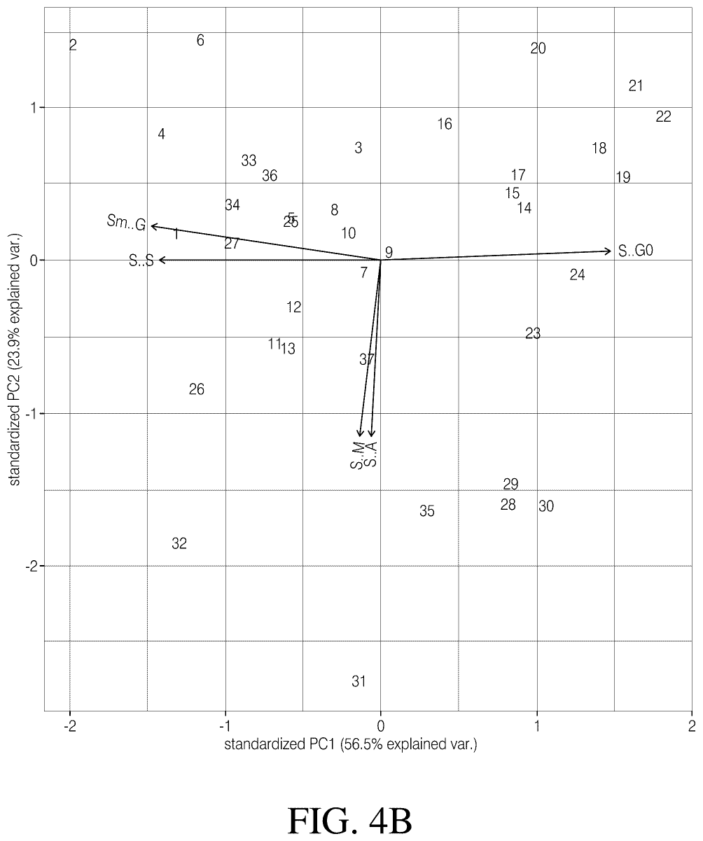

FIGS. 4-9 are graphs showing non-limiting exemplary embodiments for using multiple biophysical attributes to predict related functional response, such as an ADCC response in an example with a model built using a three-molecule (therapeutic mAb 1, 2, 3) dataset. Using this dataset, each possible component of the workflow is outlined in FIG. 3 and in detail below. Note again that FIG. 3 serves as an example workflow for predicting functional activity based on a selected combination of related biophysical attributes and, as such, each and every component illustrated therein need not be included for all embodiments.

The workflow 300 in FIG. 3 can include, at step 310, receiving raw data comprising data related to an initial set of predictors labeled with corresponding functional response. For example, the raw data can be a dataset comprising HILIC glycan data sums (Sum of asialo-agalacto-fucosylated biantennary oligosaccharide (G0F), Sum of afucosylation, Sum of galactosylation, Sum of Mannose, and Sum of Sialylation in the Fc region of three antibody molecules) and ADCC functional results from a combination of three Chinese hamster ovary (CHO)-derived antibody molecules, including three IgG1 therapeutics (therapeutic mAb 1, 2, 3).

At step 310, the data containing the desired predictors (e.g., HILIC Glycan structure relative percent area values) and response (e.g., in vitro ADCC normalized percent value) was loaded into the R script as a .csv file. This file is manually generated by the user and formatting instructions are included in the script. After the data has been loaded in, the user defined the type of model the user would like to run.

The workflow 300 can include, at step 320, data cleaning. The step 320 can include formatting and loading the desired data. In various embodiments, the raw input data can also be cleaned by cither omitting missing data or imputing the mean value for the predictor in its place, depending on user preference. As used herein, “data1.0” corresponds to the full cleaned dataset of predictors and response for the correlation plot, PC A analysis (response is removed by the code here), feature ranking, and/or feature selection.

The workflow 300 can include, at step 330, visualizing correlations between different variables in the cleaned data and sample distribution. The example presented herein omitted all missing data for data cleaning. The cleaned data was used to graph a correlation plot of all the variables compared (FIG. 4A) and a principal component analysis (PCA) was carried out to visualize variation within the samples and further determine correlations between the predictors (FIG. 4B). FIG. 4A indicates correlation between the compared variables (including predictors and response). FIG. 4B illustrates a PCA biplot, in which the first two principal components (PCs) are represented by the x and y-axis and illustrate the majority of the variance within the data. These PCs are linear combinations of the predictors, which are represented as arrows in the plot.

The workflow 300 can include, at step 340, determination of variable importance and feature selection. At step 340, the cleaned data was used to perform a feature selection to identify and select which subset of predictors will train the most accurate predictive model, measured using the root mean squared error of prediction (RMSEP). As used herein, “data2.0” corresponds to the dataset of optimal subset of predictors and the response that is used to validate the model and estimate prediction performance on unseen samples (train/test split data) and to train a full model that will be used to predict desired samples (full data).

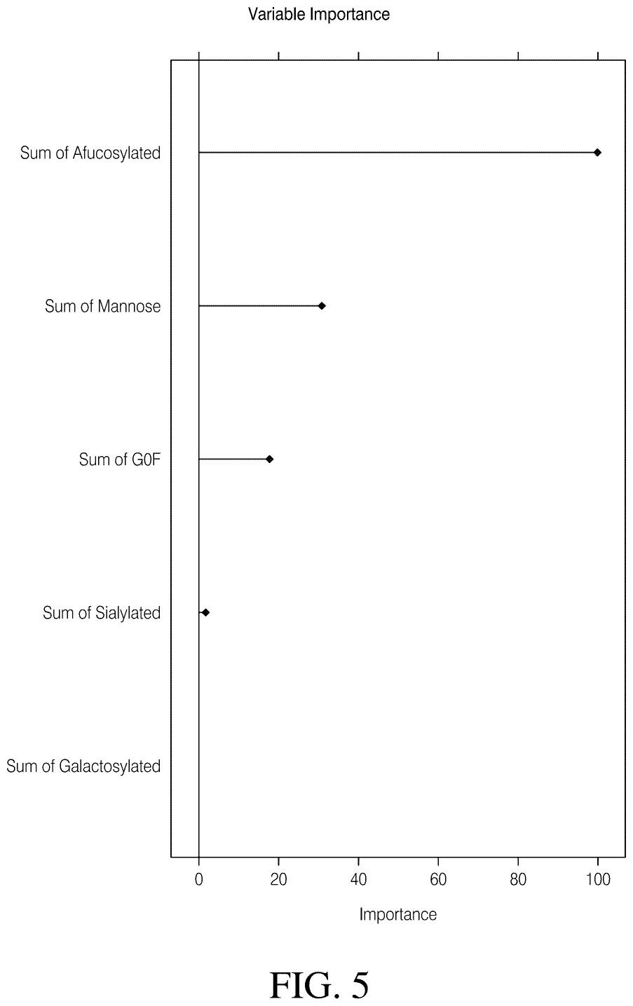

Initially, each predictor was ranked by calculating the relative contribution of each predictor to the model for variable importance ranking (FIG. 5).

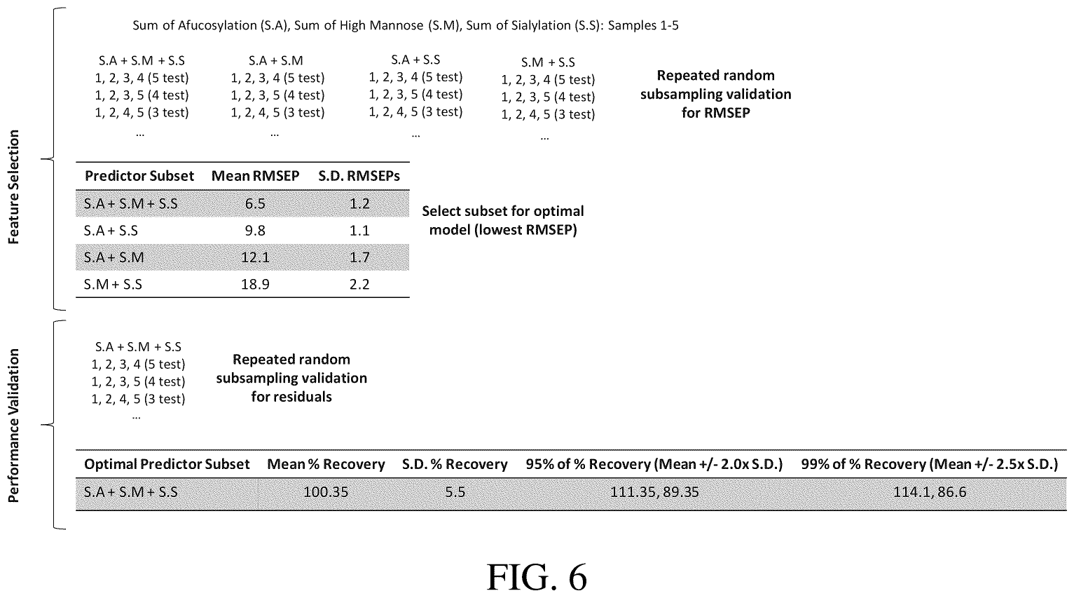

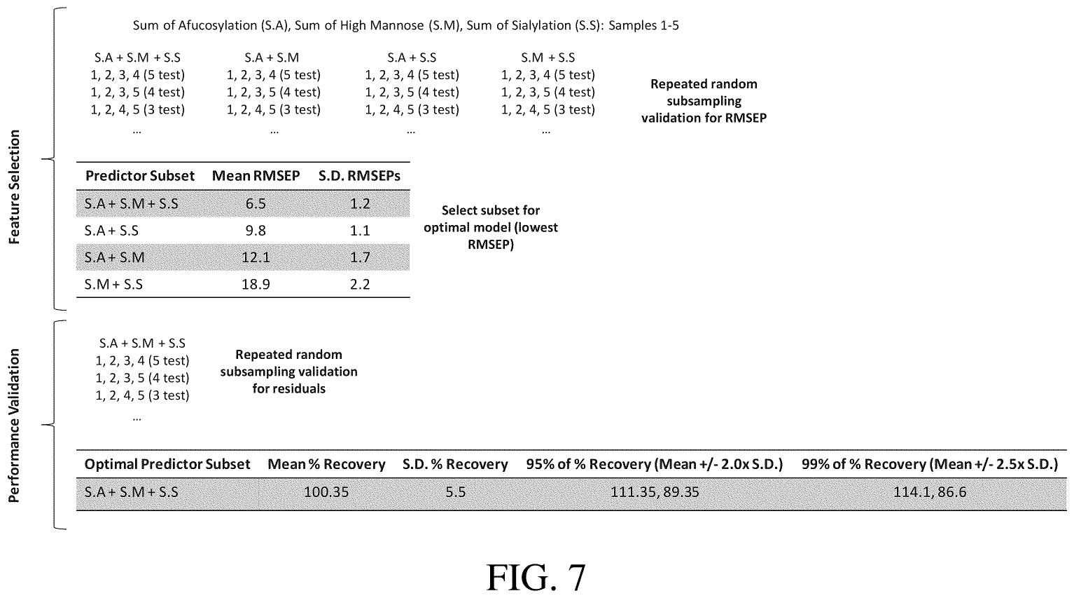

After variable importance ranking, feature selection was carried out through two different methods. Feature selection via the first method is more thorough at the expense of computational effort and time (the top part of FIG. 6) while feature selection via the second method is more efficient at the expense of being less exhaustive (the top part of FIG. 7).

In the first feature selection method, every single possible combination of predictors undergoes repeated random subsampling validation, whereby the data is split into a train set used to build a model and a test set used to validate the model. The trained model predicts values for the test set sample responses, which are directly compared to the actual measured values to calculate the RMSEP of that model. This is carried out through a user-defined number of iterations of random train and test set splits for every combination of predictors. The subset of predictors yielding models with the best average predictive accuracy (lowest average RMSEP) is then automatically selected to move forward. This method can be computationally taxing because every combination of predictors undergoes random subsampling validation for the user-defined number of iterations.

In the second feature selection method, the number of combinations of predictors is initially reduced by running a preliminary 5-fold cross-validation on every combination of predictors. Rather than training and validating multiple models on different iterations of randomized train and test set splits, the data is split only once into 5 different groups. All the groups except for one are used as a training set to fit a model, which is then evaluated using the remaining group as a test set. This process is carried out until each group serves as a test set once, and the average performance for prediction of the test sets is reported.

Given that only one data split is used to train and validate a single model, this process in the second feature selection method can be much less time consuming than repeated random subsampling validation in the first feature selection method. The top performing percentage of predictor subsets for 5-fold cross-validation automatically move on to repeated random subsampling validation.

When running the workflow in FIG. 3 using identical hardware for the three-molecule dataset containing five HILIC Glycan predictors, the first feature selection method took 21 minutes and 31 seconds and feature selection via the second feature selection method took 1 minute and 54 seconds. Depending on the requirements or constraints of the particular application, either feature selection method can be used in methods and systems described herein.

Using either feature selection methods was able to identify the same optimal subset of predictors. With more predictors, the total number of possible combinations of these predictors can increase drastically, and also the computational time in the first or second feature selection method can increase.

The workflow 300 can include, at step 350, cleaning feature-selected data. The workflow 300 can include, at step 360, modeling data split selection by selecting a splitting method to split that cleaned data into training data and test data for model performance validation at step 370.

The workflow 300 can include, at step 370, validation of model performance. After either feature selection method was used to select the optimal subset of predictors, repeated random subsampling was used on the data from this selected optimal subset to estimate the performance of a single model built on the entirety of this data in predicting unseen samples (performance validation in the bottom part of FIG. 6 and FIG. 7).

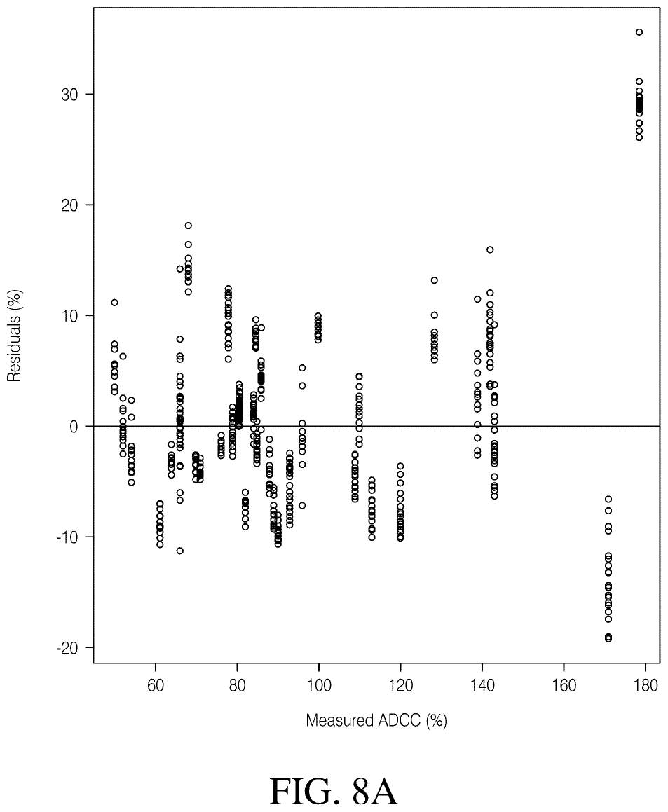

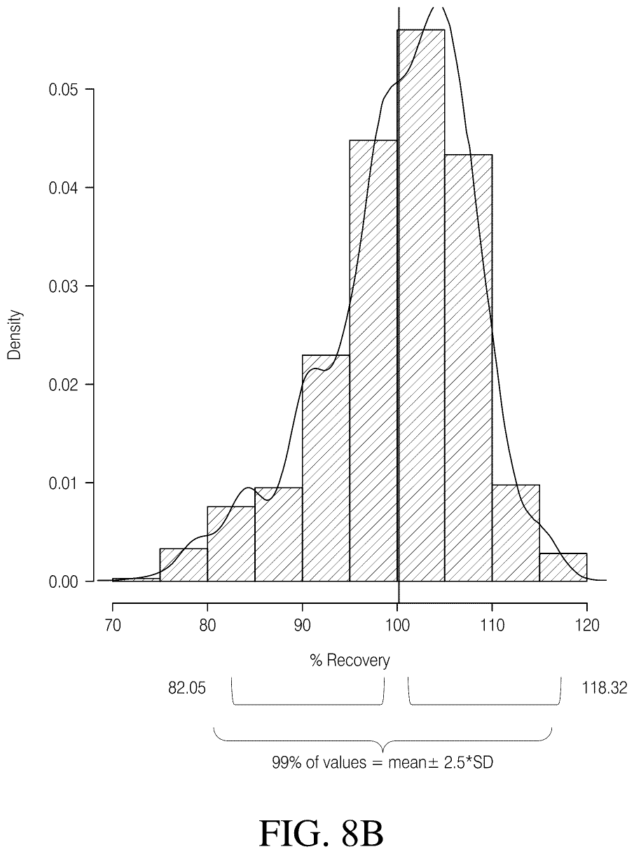

At step 370, model performance in repeated random subsampling is compounded via a residual analysis of all predicted test set samples (FIG. 8A). Here, the residuals arc the difference between the measured and predicted ADCC values and are a direct measurement of the how far the model predictions were from the true value. The residual plot for an ideal model has a high density of points close to zero (small difference between predicted and measured values) and is symmetric about zero (homoscedastic). Homoscedasticity of residuals implies that the model is uniformly predicting points, that is, it performs equally regardless of the magnitude of the actual response value.

The workflow 300 can include, at step 380, prediction of performance for the trained model. At step 380, the predictive accuracy of the model after repeated random subsampling can be reported via % recovery (predicted value/measured value*100) in order to capture the relative error of prediction for each sample, and see whether errors fit within established tolerances, typically 80-120% recovery. For a normally distributed set of values, 95% of the values falls within two standard deviations of the mean value and 99% of the values fall within 2.5 standard deviations of the mean value. This statistical approximation, known as the empirical rule, can be leveraged to predict the model performance for desired samples by reporting an estimated range of values that the majority of % recovery values (95% and 99% of values) fall within, in other words, an approximation of how far off the majority of the model predictions for ADCC can be from the actual measured value for the samples in the data.

Thus, the predictive power of the three-molecule model is within an 80-120% recovery range (99% confidence interval). The ability to consistently predict within a range generally accepted for assay qualification bolsters this model's usefulness in situations where prior glycan and ADCC data is limited or not available for a newer molecular entity of a similar format (e.g., IgGl mAb).

The population of values are desirable to be normally distributed so that the performance prediction can be held true. Therefore, a qualitative analysis via a probability density plot was performed to confirm the values for % recovery for all predicted test set samples are normally distributed (FIG. 8B). FIG. 8B also shows that the percentage of recovery (which equals to predicted ADCC/measured ADCC*100) is between about 80% to about 120%.

After estimating the performance of the final model on predicting response for disabled samples (e.g., unseen data), the actual model is built by training on the full data for the optimal predictors (no train/test split). Predictions can be made for any sample with an identical set of measured predictors as was used to train the final model.

In addition to the analysis of a model using three molecules (therapeutic mAb 1, 2, 3), as detailed above in FIGS. 4-9, several other models were generated from combinations of the three-molecule data. The validation metrics for each of these models are presented in Table 1. In Table 1, the key is as follows: Sum of G0F (G0F+G0F−N)=S.G0F, Sum of Afucosylation (G0−N+G0+G1)=S.A., Sum of Galactosylation (G1F+G2F+G1)=S.G., Sum of Mannose (M5+M6+M7+M8)=S.M., Sum of Sialylation (G1S1F+G2S1F+G2S2F)=S.S. Note: Repeated random subsampling was carried out over 100 iterations of train and test set splits (80/20 split of full data), and terms in [brackets] correspond to data where a single outlier for therapeutic mAb 1 was removed.

TABLE 1

| Molecules | | Mean | 95% of % | 99% of % |
|---|---|---|---|---|
| Used | Optimal Subset Attributes | RMSEP | Recovery | Recovery |
| Therapeutic | S.GOF, S.A., S.G., S.M., S.S. | 7.52 | 114.69, 85.67 | 118.32, 82.05 |
| mAb 1 | (N = 37) | [5.98] | [115.27, 87.44] | [118.75, 83.96] |
| | [S.G0F, S.A., S.M., S.S.] | | | |
| Therapeutic | S.A., S.G., S.S. | 7.73 | 114.46, 86.65 | 117.93, 83.17 |
| mAb 2 | (N = 52) | | | |
| Therapeutic | S.GOF, S.A., S.M., S.S. | 4.14 | 110.80, 89.73 | 113.44, 87.09 |
| mAb 3 | (N = 43) | | | |
| Therapeutic | S.A., S.G., S.S. | 8.65 | 117.04, 84.53 | 121.10. 80.47 |
| mAb 1 + 2 | (N = 89) | [7.66] | [116.39, 85.02] | [120.31, 81.10] |
| | [S.A., S.G., S.S.] | | | |
| Therapeutic | S.GOF, S.A., S.G., S.M. | 6.87 | 115.01.85.54 | 118.69, 81.86 |
| mAb 1 + 3 | (N = 80) | [5.66] | [113.79, 86.86] | [117.15, 83.50] |
| | [S.GOF, S.A.. S.G., S.M.] | | | |
| Therapeutic | S.GOF, S.A., S.G., S.M. | 7.31 | 115.07, 85.93 | 118.72, 82.29 |
| mAb 2 + 3 | (N = 95) | | | |
| Therapeutic | S.G0F. S.A., S.G., S.M. | 8.32 | 116.62, 84.58 | 120.63, 80.58 |
| mAb 1 + 2 + 3 | (N = 132) | [7.39] | [116.01, 85.43] | [119.83, 81.61] |
| | [S.G0F, S.A., S.G., S.M., S.S.] | | | |

The performance of this data modelling workflow in FIG. 3 was compared to a commonly used data analysis technique: linear regression using a known attribute that is strongly linearly correlated to response. In this case, the sum of afucosylation vs. ADCC response was used (Table 2).

The PLS model has a smaller range of values that covers 99% of % recovery compared to the linear regression in every case except for therapeutic mAb 1+2+3, where the range of values are nearly identical. Importantly, all of the individual molecule in PLS models are safely within 80-120% recovery for 99% of the % recovery values for the predicted samples, whereas therapeutic mAb 1 and 3 deviate from this threshold significantly in the linear regression.

As shown in Table 2 the PLS model performs as well as linear regression (shown in brackets) in datasets that have a strong univariate linear correlation that dictates the majority of the response behavior, but performs much better when correlations behave non-linearly or if there are significant correlations between multiple predictors and the response. Either way, the PLS model is more robust and ultimately more practical to use for this manner of data analysis. It is worth noting that in most cases, the threshold of success in the predictive accuracy of the model will be defined by the user and the context of the analysis.

In the case presented in Table 2, a % recovery range between 80% and 120% was used as the accepted level of error due to this range being a generally accepted margin of error in the qualification of analytical assays. Using this metric, we can estimate that the PLS model predicts the majority (99%) of unseen samples satisfactorily (within 80-120% recovery).

TABLE 2
Output for linear regression models
trained on Therapeutic mAbdata

| | Mean | 95% of % | 99% of % |
|---|---|---|---|
| Molecules Used | RMSEP | Recovery | Recovery |
| Therapeutic mAb 1 | 8.55 | 118.24, 82.49 | 122.71, 78.02 |
| | [7.47] | [118.55, 81.55] | [123.18, 76.92] |
| Therapeutic mAb 2 | 7.80 | 114.23, 86.58 | 117.68, 83.13 |
| Therapeutic mAb 3 | 8.95 | 124.45.77.98 | 130.26, 72.17 |
| Therapeutic mAb 1 + 2 | 8.69 | 116.66, 85.01 | 120.61, 81.05 |
| | [7.59] | [115.93, 85.45] | [119.74.81.64] |
| Therapeutic mAb 1 + 3 | 15.47 | 140.98, 63.48 | 150.66, 53.80 |
| | [14.61] | [139.60, 64.57] | (148.97.55.19] |
| Therapeutic mAb 2 + 3 | 12.01 | 125.04, 77.16 | 131.03, 71.18 |
| Therapeutic mAb | 13.81 | 134.30, 70.85 | 142.23, 62.92 |
| 1 + 2 + 3 | [13.29] | [134.00, 71.14] | [141.85, 63.29] |

Note:
terms in brackets correspond to data where the single outlier for therapeutic mAb 1 was removed.

Finally, the performances of the PLS model compared to random forest model and support vector machines (two widely used machine learning algorithms) were tested (Table 3). Within the context of this data set (size, complexity, etc.) the PLS model performed equally well or better than the other models when comparing the mean RMSEP, although this is to be expected as more complex machine learning algorithms tend to underperform with smaller data sets

TABLE 3
Differences in performance between models

| Model Used | Package(s) | Optimal Subset | Mean | 95% of % | 99% of % |
|---|---|---|---|---|---|
| (Method) | (Ver.) | Attributes | RMSEP | Recovery | Recovery |
| Partial Least | pls (2.7-3) | S.G0F, S.A., | 8.32 | 116.62, | 120.63. |
| Square | | S.G., S.M. | | 84.58 | 80.58 |
| (kernelpls) | | (N = 132) | | | |
| Random Forest | E1071 (1.7-4), | S.A., S.G., S.M. | 9.20 | 119.68, | 124.40, |
| (ranger) | ranger(0.12.1), | (N = 132) | | 81.85 | 77.12 |
| | dplyr (1.0.2) | | | | |
| Support Vector | LiblineaR | S.A., S.G., S.M. | 8.34 | 116.04, | 120.02, |
| Machine | (2.10-8) | (N = 132) | | 84.21 | 80.24 |
| (svmLinear3) | | | | | |

Note:
All models were built using three-molecule database, all models use 100 iterations for repeated random subsampling with an 80/20 train/test split.

III.B. Methods

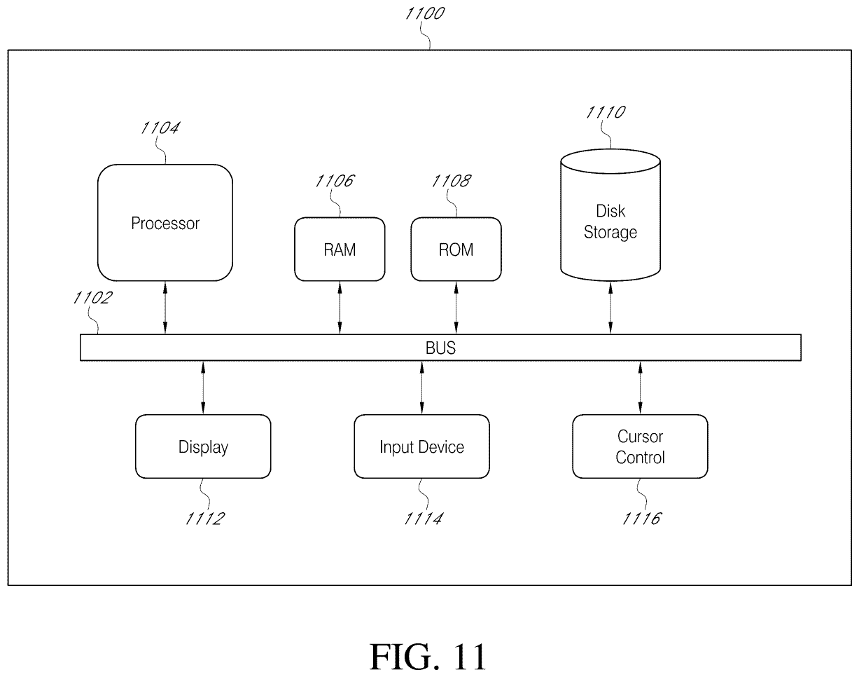

In accordance with various embodiment, various exemplary methods are provided for predicting functional activity based on related biophysical attributes. The methods can incorporate one or more features of the workflow 100, 200, or 300 (interchangeably, in any combination), and can be implemented via computer software or hardware, or a combination thereof, for example, as exemplified in FIG. 10 or FIG. 11. The methods can also be implemented on a computing device/system that can include a combination of engines for detecting candidates for target binding. In various embodiments, the computing device/system can be communicatively connected to one or more of a data source, data modeling analyzer, and display device via a direct connection or through an internet connection.

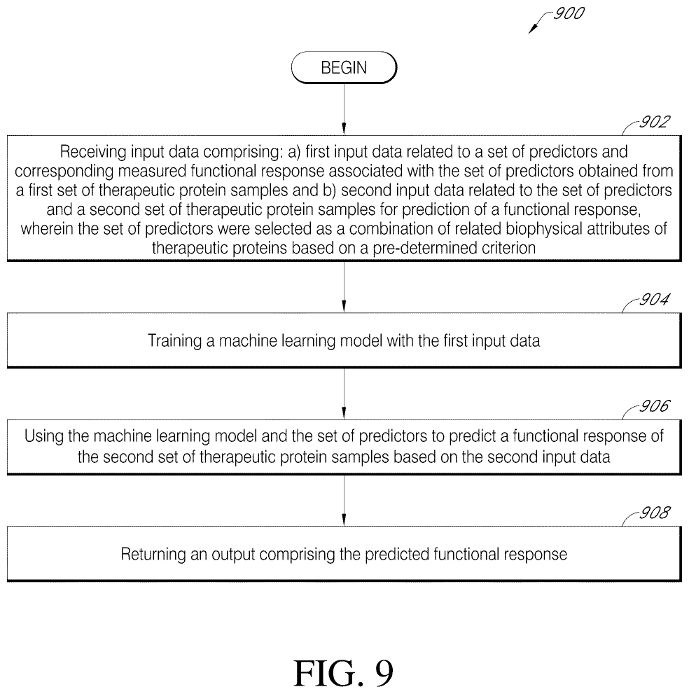

Referring now to FIG. 9, a flowchart illustrating a non-limiting example method 900 for predicting functional activity based on related biophysical attributes, in accordance with various embodiments. The method 900 can comprise, at step 902, receiving input data.

The input data can include first input data related to a set of predictors and corresponding measured functional response (e.g., measured antibody-dependent cellular cytotoxicity (ADCC) response) associated with the set of predictors obtained from a first set of therapeutic protein (e.g., antibody) samples. The input data can further include second input data related to the set of predictors and a second set of therapeutic protein (e.g., antibody) samples for prediction of the functional response (e.g., ADCC response). In various embodiments, the set of predictors were selected as a combination of related biophysical attributes of therapeutic proteins based on a pre-determined criterion, such as a combination of a degree of afucosylation and one or more additional glycosylation attributes of antibodies. For example, the one or more additional glycosylation attributes of antibodies comprise galactosylation, sialylation, glycan chain length, glycan building block type, high molecular weight forms, and forms of antibodies missing N-glycan chains, or any combination thereof. The first set of therapeutic protein (e.g., antibody) samples or the second set of therapeutic protein (e.g., antibody) samples can comprise monoclonal antibody samples.

The method 900 can comprise, at step 904, training a machine learning model with the first input data. The step 904 can include selecting the set of predictors from a plurality of combinations of the related biophysical attributes of therapeutic proteins, such as, for example, the degree of afucosylation and/or the one or more additional glycosylation attributes of antibodies. Selecting the set of predictors can include repeated random subsampling validation or cross-validation using a pre-defined split of the first input data, such as a five-fold cross-validation.

The step 904 can further include selecting the machine learning model. The machine learning model can be selected if the machine learning model is determined to have a model performance that meets a predefined threshold using the first input data and the set of predictors. The machine learning model can be a model based on, for example, partial least square, random forest, support vector machine, Naive Bayes, KNN, Generalized additive model, logistic regression, gradient boosting, or lasso.

The method 900 can comprise, at step 906, predicting a functional response (e.g., ADCC response) of the second set of therapeutic protein (e.g., antibody) samples based on the second input data. The predicting can be done using the machine learning model and the set of predictors.

The method 900 can comprise, at step 908, returning an output comprising the predicted ADCC response. The method 900 can further comprise selecting a therapeutic candidate from the second set of therapeutic protein (e.g., antibody) samples based on the predicted functional response (e.g., predicted ADCC response). The method 900 can further comprise validating a therapeutic efficacy of the therapeutic candidate. The method 900 can further comprise developing a therapeutic compositing comprising the therapeutic candidate. The prediction engine 1012 can predict ADCC response using the machine learning model and the set of predictors.

III.C. Systems

In various embodiments, any methods for predicting functional activity based on a selected combination of related biophysical attributes or as exemplified in workflow 100, 200, or 300 can be implemented via software, hardware, firmware, or a combination thereof, such as described in FIG. 10. FIG. 10 illustrates a non-limiting example system configured to predict functional activity based on a selected combination of related biophysical attributes, in accordance with various embodiments. The system 1000 can include various combinations of features, whether it be more or less features than that are illustrated in FIG. 10. As such, FIG. 10 simply illustrates one example of a possible system.

The system 1000 includes a data collection unit 1002, a data storage unit 1004, a computing device/analytics server 1006, a display 1014, and a validation unit 1016. The data collection unit 1002 can be communicatively connected to and can send datasets to the data storage unit 1004 by way of a serial bus (if both form an integrated instrument platform) or by way of a network connection (if both are distributed/separate devices). The generated datasets are stored in the data storage unit 1004 for subsequent processing. In various embodiments, one or more raw datasets can also be stored in the data storage unit 1004 prior to processing and analyzing. Accordingly, in various embodiments, the data storage unit 1004 can be configured to store datasets of the various embodiments herein that correspond to several sets of therapeutic protein (e.g., antibody) samples. In various embodiments, the processed datasets can be fed to the computing device/analytics server 1006 in real-time for further downstream analysis.

The data storage unit 1004 can be communicatively connected to the computing device/analytics server 1006. In various embodiments, the data storage unit 1004 and the computing device/analytics server 1006 can be part of an integrated apparatus. In various embodiments, the data storage unit 1004 can be hosted by a different device than the computing device/analytics server 1006. In various embodiments, the data storage unit 1004 and the computing device/analytics server 1006 can be part of a distributed network system. In various embodiments, the computing device/analytics server 1006 can be communicatively connected to the data storage unit 1004 via a network connection that can be either a “hardwired” physical network connection (e.g., Internet, LAN, WAN, VPN, etc.) or a wireless network connection (e.g., Wi-Fi, WLAN, etc.). The computing device/analytics server 1006 can be a workstation, mainframe computer, distributed computing node (part of a “cloud computing” or distributed networking system), personal computer, mobile device, etc., according to various embodiments. The computing device/analytics server 1006 can be a client computing device. In various embodiments, the computing device/analytics server 1006 can be a personal computing device having a web browser (e.g., INTERNET EXPLORER™, FIREFOX™, SAFARI™, etc.) that can be used to control the operation of the data collection unit 1002, data storage unit 1004, display 1014, and validation unit 1016.

The computing system such as computer device/analytics sever 1006 is configured to host one or more feature selection engines 1008, one or more training engines 1010, and/or one or more prediction engines 1012, according to various embodiments. The feature selection engine 1008 is configured to select the set of predictors from a plurality of combinations of the degree of afucosylation and the one or more glycosylation attributes of antibodies. In various embodiments, the one or more glycosylation attributes of antibodies comprise galactosylation, sialylation, glycan chain length, glycan building block type, high molecular weight forms, and forms of antibodies missing N-glycan chains, or any combination thereof. The training engine 1010 can be configured to train a machine learning model, for example, with the first input data. The prediction engine 1012 can be configured to predict ADCC response of the second set of therapeutic protein (e.g., antibody) samples based on the second input data. The prediction engine 1012 can predict ADCC response using the machine learning model and the set of predictors. The prediction engine 1012 can be further configured to select therapeutic candidates from the second set of therapeutic protein (e.g., antibody) samples based on the prediction of functional response. The system 1000 further comprises a validation unit 1016 configured to validate desired functional response of the selected candidates.

During the time when the computing device/analytics server 1006 is receiving and processing data from the data storage unit 1004 or after the processing is done, an output of the results can be displayed as a result or summary on a display 1014 that is communicatively connected to the computing device/analytics server 1006. The display 1014 can be a client computing device or a client terminal. The display 1014 can be a personal computing device having a web browser (e.g., INTERNET EXPLORER™, FIREFOX™, SAFARI™, etc.) that can be used to control the operation of the operation of the data collection unit 1002, data storage unit 1004, feature selection engine 1008, training engines 1010, prediction engines 1012, and display 1014.

It should be appreciated that the various engines can be combined or collapsed into a single engine, component or module, depending on the requirements of the particular application or system architecture. Engines 1008/1010/1012 can comprise additional engines or components as needed by the particular application or system architecture.

### IV. Computer-Implemented System

In various embodiments, any methods for predicting functional activity based on a selected combination of related biophysical attributes or as exemplified in workflow 100, 200, or 300 can be implemented via software, hardware, firmware, or a combination thereof, such as described in FIG. 10 or FIG. 11.

That is, as depicted in FIG. 10, the methods disclosed herein can be implemented on a computer system such as computer system 1000 (e.g., a computing device/analytics server). The computer system 1000 can include a computing device/analytics server 1006, which can be communicatively connected to a data storage 1004 and a display system 1014 via a direct connection or through a network connection (e.g., LAN, WAN, Internet, etc.). It should be appreciated that the computer system 1000 depicted in FIG. 10 can comprise additional engines or components as needed by the particular application or system architecture.

FIG. 11 is a block diagram illustrating a computer system 1100 upon which embodiments of the present teachings may be implemented. In various embodiments of the present teachings, computer system 1100 can include a bus 1102 or other communication mechanism for communicating information and a processor 1104 coupled with bus 1102 for processing information. In various embodiments, computer system 1100 can also include a memory, which can be a random-access memory (RAM) 1106 or other dynamic storage device, coupled to bus 1102 for determining instructions to be executed by processor 1104. Memory can also be used for storing temporary variables or other intermediate information during execution of instructions to be executed by processor 1104. In various embodiments, computer system 1100 can further include a read only memory (ROM) 1108 or other static storage device coupled to bus 1102 for storing static information and instructions for processor 1104. A storage device 1110, such as a magnetic disk or optical disk, can be provided and coupled to bus 1102 for storing information and instructions.

In various embodiments, processor 1104 can be coupled via bus 1102 to a display 1012, such as a cathode ray tube (CRT) or liquid crystal display (LCD), for displaying information to a computer user. An input device 1114, including alphanumeric and other keys, can be coupled to bus 1002 for communication of information and command selections to processor 1104. Another type of user input device is a cursor control 1116, such as a mouse, a trackball or cursor direction keys for communicating direction information and command selections to processor 1104 and for controlling cursor movement on display 1112.

Consistent with certain implementations of the present teachings, results can be provided by computer system 1100 in response to processor 1104 executing one or more sequences of one or more instructions contained in memory 1106. Such instructions can be read into memory 1106 from another computer-readable medium or computer-readable storage medium, such as storage device 1110. Execution of the sequences of instructions contained in memory 1106 can cause processor 1104 to perform the processes described herein. In various embodiments, hard-wired circuitry can be used in place of or in combination with software instructions to implement the present teachings. Thus, implementations of the present teachings arc not limited to any specific combination of hardware circuitry and software.

The term “computer-readable medium” (e.g., data store, data storage, etc.) or “computer-readable storage medium” as used herein refers to any media that participates in providing instructions to processor 1104 for execution. Such a medium can take many forms, including but not limited to, non-volatile media, volatile media, and transmission media. Examples of non-volatile media can include, but are not limited to, dynamic memory, such as memory 1106. Examples of transmission media can include, but are not limited to, coaxial cables, copper wire, and fiber optics, including the wires that comprise bus 1102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 1104 of computer system 1100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 1000 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPG As), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as R, C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1100, whereby processor 1104 would execute the analyses and determinations provided by these engines, subject to instructions provided by any one of, or a combination of, memory components 1106/1108/1110 and user input provided via input device 1114.

The term “computer-readable medium” (e.g., data store, data storage, etc.) or “computer-readable storage medium” as used herein refers to any media that participates in providing instructions to processor 1104 for execution. Such a medium can take many forms, including but not limited to, non-volatile media, volatile media, and transmission media. Examples of non-volatile media can include, but are not limited to, optical, solid state, magnetic disks, such as storage device 1110. Examples of volatile media can include, but are not limited to, dynamic memory, such as memory 1106. Examples of transmission media can include, but are not limited to, coaxial cables, copper wire, and fiber optics, including the wires that comprise bus 1102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1104 of computer system 1100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flow charts, diagrams and accompanying disclosure can be implemented using computer system 1200 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPG As), processors, controllers, micro-controllers, microprocessors, electronic devices, other electronic units designed to perform the functions described herein, or a combination thereof.

Digital Processing Device

In various embodiments, the systems and methods described herein can include a digital processing device or use of the same. In various embodiments, the digital processing device can include one or more hardware central processing units (CPUs) or general-purpose graphics processing units (GPGPUs) that carry out the device's functions. In various embodiments, the digital processing device further comprises an operating system configured to perform executable instructions. In various embodiments, the digital processing device can be optionally connected a computer network. In various embodiments, the digital processing device can be optionally connected to the Internet such that it accesses the World Wide Web. In various embodiments, the digital processing device can be optionally connected to a cloud computing infrastructure. In various embodiments, the digital processing device can be optionally connected to an intranet. In various embodiments, the digital processing device can be optionally connected to a data storage device.

In accordance with various embodiments, suitable digital processing devices can include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. Those of ordinary skill in the art will recognize that many smartphones are suitable for use in the system described herein. Those of ordinary skill in the art will also recognize that select televisions, video players, and digital music players with optional computer network connectivity are suitable for use in the system described herein. Suitable tablet computers include those with booklet, slate, and convertible configurations, known to those of ordinary skill in the art.

In various embodiments, the digital processing device includes an operating system configured to perform executable instructions. The operating system can be, for example, software, including programs and data, which manages the device's hardware and provides services for execution of applications. Those of ordinary skill in the art will recognize that suitable server operating systems include, by way of non-limiting examples, FreeBSD, OpenBSD, Net-BSD, Linux, Apple® Mac OS X Server®, Oracle® Solaris®, Windows Server®, and Novell® NetWare®. Those of ordinary skill in the art will recognize that suitable personal computer operating systems include, by way of non-limiting examples, Microsoft® Windows®, Apple® Mac OS X®, UNIX®, and UNIX-like operating systems such as GNU/Linux®. In various embodiments, the operating system is provided by cloud computing. Those of ordinary skill in the art will also recognize that suitable mobile smart phone operating systems include, by way of non-limiting examples, Nokia® Symbian® OS, Apple® iOS®, Research In Motion® Black-Berry OS®, Google® Android®, Microsoft® Windows Phone® OS, Microsoft® Windows Mobile® OS, Linux®, and Palm® WebOS®.

In various embodiments, the device includes a storage and/or memory device. The storage and/or memory device is one or more physical apparatuses used to store data or programs on a temporary or permanent basis. In various embodiments, the device is volatile memory and requires power to maintain stored information. In various embodiments, the device is non-volatile memory and retains stored information when the digital processing device is not powered. In various embodiments, the non-volatile memory comprises flash memory. In various embodiments, the non-volatile memory comprises dynamic random-access memory (DRAM). In various embodiments, the non-volatile memory comprises ferroelectric random access memory (FRAM). In various embodiments, the non-volatile memory comprises phase-change random access memory (PRAM). In various embodiments, the device is a storage device including, by way of non-limiting examples, CD-ROMs, DVDs, flash memory devices, magnetic disk drives, magnetic tapes drives, optical disk drives, and cloud computing-based storage. In various embodiments, the storage and/or memory device is a combination of devices such as those disclosed herein.

In various embodiments, the digital processing device includes a display to send visual information to a user. In various embodiments, the display is a cathode ray tube (CRT). In various embodiments, the display is a liquid crystal display (LCD). In various embodiments, the display is a thin film transistor liquid crystal display (TFT-LCD). In various embodiments, the display is an organic light emitting diode (OLED) display. In various embodiments, on OLED display is a passive-matrix OLED (PMOLED) or active-matrix OLED (AMOLED) display. In various embodiments, the display is a plasma display. In various embodiments, the display is a video projector. In various embodiments, the display is a combination of devices such as those disclosed herein.

In various embodiments, the digital processing device includes an input device to receive information from a user. In various embodiments, the input device is a keyboard. In various embodiments, the input device is a pointing device including, by way of non-limiting examples, a mouse, trackball, track pad, joystick, game controller, or stylus. In various embodiments, the input device is a touch screen or a multi-touch screen. In various embodiments, the input device is a microphone to capture voice or other sound input. In various embodiments, the input device is a video camera or other sensor to capture motion or visual input. In various embodiments, the input device is a Kinect, Leap Motion, or the like. In various embodiments, the input device is a combination of devices such as those disclosed herein.

Non-Transitory Computer Readable Storage Medium

In various embodiments, and as stated above, the systems and methods disclosed herein can include, and the methods herein can be run on, one or more non-transitory computer readable storage media encoded with a program including instructions executable by the operating system of an optionally networked digital processing device. In various embodiments, a computer readable storage medium is a tangible component of a digital processing device. In various embodiments, a computer readable storage medium is optionally removable from a digital processing device. In various embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In various embodiments, the program and instructions are permanently, substantially permanently, semi-permanently, or non-transitorily encoded on the media.

Computer Program

In various embodiments, the systems and methods disclosed herein can include at least one computer program or use at least one computer program. A computer program includes a sequence of instructions, executable in the digital processing device's CPU, written to perform a specified task. Computer readable instructions may be implemented as program modules, such as functions, objects, Application Programming Interfaces (APis), data structures, and the like, that perform particular tasks or implement particular abstract data types. Those of ordinary skill in the art will recognize that a computer program may be written in various versions of various languages.

The functionality of the computer readable instructions may be combined or distributed as desired in various environments. In various embodiments, a computer program comprises one sequence of instructions. In various embodiments, a computer program comprises a plurality of sequences of instructions. In various embodiments, a computer program is provided from one location. In various embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

Web Application

In various embodiments, a computer program includes a web application. Those of ordinary skill in the art will recognize that a web application, in various embodiments, utilizes one or more software frameworks and one or more database systems. In various embodiments, a web application is created upon a software framework such as Microsoft® .NET or Ruby on Rails (RoR). In various embodiments, a web application utilizes one or more database systems including, by way of non-limiting examples, relational, non-relational, object oriented, associative, and XML database systems.

In various embodiments, suitable relational database systems include, by way of non-limiting examples, Microsoft® SQL Server, mySQL™, and Oracle®. Those of ordinary skill in the art will also recognize that a web application, in various embodiments, is written in one or more versions of one or more languages. A web application may be written in one or more markup languages, presentation definition languages, client-side scripting languages, server-side coding languages, data-base query languages, or combinations thereof. In various embodiments, a web application is written to some extent in a markup language such as Hypertext Markup Language (HTML), Extensible Hypertext Markup Language (XHTML), or extensible Markup Language (XML). In various embodiments, a web application is written to some extent in a presentation definition language such as Cascading Style Sheets (CSS).

In various embodiments, a web application is written to some extent in a client-side scripting language such as Asynchronous Javascript and XML (AJAX), Flash® Actionscript, Javascript, or Silverlight®. In various embodiments, a web application is written to some extent in a server-side coding language such as Active Server Pages (ASP), ColdFusion®, Perl, Java™, JavaServer Pages (JSP), Hypertext Preprocessor (PHP), Python™ Ruby, Tel, Smalltalk, WebDNA®, or Groovy. In various embodiments, a web application is written to some extent in a database query language such as Structured Query Language (SQL). In various embodiments, a web application integrates enterprise server products such as IBM® Lotus Domino®. In various embodiments, a web application includes a media player element. In various embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

Mobile Application

In various embodiments, a computer program includes a mobile application provided to a mobile digital processing device. In various embodiments, the mobile application is provided to a mobile digital processing device at the time it is manufactured. In various embodiments, the mobile application is provided to a mobile digital processing device via the computer network described herein.

A mobile application can be created by techniques known to those of ordinary skill in the art using hardware, languages, and development environments known to the art. Those of ordinary skill in the art will recognize that mobile applications can be written in several languages. Suitable programming languages include, by way of non-limiting examples, C, C++, C #, Objective-C, Java™, JavaScript, Pascal, Object Pascal, Rust, Python™, Ruby, VB.NET, WML. and XHTML/HTML with or without CSS, or combinations thereof.

Suitable mobile application development environments arc available from several sources. Commercially available development environments include, by way of non-limiting examples, AirplaySDK, alcheMo, Appcelera-tor®, Celsius, Bedrock, Flash Lite, .NET Compact Frame-work, Rhomobile, and WorkLight Mobile Platform. Other development environments are available without cost including, by way of non-limiting examples, Lazarus, Mobi-Flex, MoSync, and Phonegap. Also, mobile device manufacturers distribute software developer kits including, by way of non-limiting examples, iPhone and iPad (iOS) SDK, Android™ SDK, BlackBerry® SDK. BREW SDK, Palm® OS SDK, Symbian SDK, webOS SDK, and Windows® Mobile SDK.

Those of ordinary skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo DSi Shop.

Standalone Application

In various embodiments, a computer program includes a standalone application, which is a program that is run as an independent computer process, not an add-on to an existing process, e.g., not a plug-in. Those of ordinary skill in the art will recognize that standalone applications are often compiled. A compiler is a computer program(s) that transforms source code written in a programming language into binary object code such as assembly language or machine code. Suitable compiled programming languages include, by way of non-limiting examples, Rust, C, C++, Objective-C, COBOL, Delphi, Eiffel, Java™, Lisp, Python™, Visual Basic, and VB.NET, or combinations thereof. Compilation is often per-formed, at least in part, to create an executable program. In various embodiments, a computer program includes one or more executable complied applications.

Web Browser Plug-in

In various embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of ordinary skill in the art will be familiar with several web browser plug-ins including, Adobe® Flash® Player, Microsoft® Silver-light®, and Apple® QuickTime®. In various embodiments, the toolbar comprises one or more web browser extensions, add-ins, or add-ons. In various embodiments, the toolbar comprises one or more explorer bars, tool bands, or desk bands.

Those of ordinary skill in the art will recognize that several plug-in frame works are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

Software Modules

In various embodiments, the systems and methods disclosed herein include a software, server and/or database modules, or incorporate use of the same in methods according to various embodiments disclosed herein. Software modules can be created by techniques known to those of ordinary skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In various embodiments, software modules are in one computer program or application. In various embodiments, software modules are in more than one computer program or application. In various embodiments, software modules are hosted on one machine. In various embodiments, software modules are hosted on more than one machine. In various embodiments, software modules are hosted on cloud computing platforms. In various embodiments, software modules arc hosted on one or more machines in one location. In various embodiments, software modules are hosted on one or more machines in more than one location.

Databases

In various embodiments, the systems and methods disclosed herein include one or more databases, or incorporate use of the same in methods according to various embodiments disclosed herein. Those of ordinary skill in the art will recognize that many databases are suitable for storage and retrieval of user, query, token, and result information. In various embodiments, suitable databases include, by way of non-limiting examples, relational databases, non-relational databases, object oriented databases, object databases, entity-relation-ship model databases, associative databases, and XML databases. Further non-limiting examples include SQL, Postgr-eSQL, MySQL, Oracle, DB2, and Sybase. In various embodiments, a database is internet-based. In further Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In various embodiments, a database is web-based. In various embodiments, a database is cloud computing-based. In other embodiments, a database is based on one or more local computer storage devices.

Data Security

In various embodiments, the systems and methods disclosed herein include one or features to prevent unauthorized access. The security measures can, for example, secure a user's data. In various embodiments, data is encrypted. In various embodiments, access to the system requires multi-factor authentication and access control layer. In various embodiments, access to the system requires two-step authentication (e.g., web-based interface). In various embodiments, two-step authentication requires a user to input an access code sent to a user's e-mail or cell phone in addition to a username and password. In various instances, a user is locked out of an account after failing to input a proper username and password. The systems and methods disclosed herein can, in various embodiments, also include a mechanism for protecting the anonymity of users' genomes and of their searches across any genomes.

While the present teachings arc described in conjunction with various embodiments, it is not intended that the present teachings be limited to such embodiments. On the contrary, the present teachings encompass various alternatives, modifications, and equivalents, as will be appreciated by those of skill in the art.

In describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

### Recitation of Embodiments

EMBODIMENT 1: A method comprising: receiving input data comprising: a) first input data related to a set of predictors and corresponding measured functional response associated with the set of predictors obtained from a first set of therapeutic protein samples and b) second input data related to the set of predictors and a second set of therapeutic protein samples for prediction of a functional response, wherein the set of predictors were selected as a combination of related biophysical attributes of therapeutic proteins based on a pre-determined criterion; training a machine learning model with the first input data; using the machine learning model and the set of predictors to predict a functional response of the second set of therapeutic protein samples based on the second input data; and returning an output comprising the predicted functional response.

EMBODIMENT 2: The method of EMBODIMENT 1, wherein the therapeutic protein samples are antibody samples, the functional response is antibody-dependent cell-mediated cytotoxicity (ADCC) response, complement-dependent cytotoxicity (CDC) response, Fc gamma receptors (FcyR) binding or complement Clq binding, and the related biophysical attributes of therapeutic proteins comprise a degree of afucosylation and one or more additional glycosylation attributes of antibodies.

EMBODIMENT 3: The method of EMBODIMENT 2, wherein the one or more additional glycosylation attributes of antibodies comprise galactosylation, sialylation. glycan chain length, glycan building block type, and forms of antibodies missing N-glycan chains, or any combination thereof.

EMBODIMENT 4: The method of EMBODIMENTS 2 or 3, wherein the one or more additional glycosylation attributes of antibodies comprise two glycosylation attributes of antibodies.

EMBODIMENT 5: The method of any one of EMBODIMENTS 2 to 4, wherein the one or more additional glycosylation attributes of antibodies comprise galactosylation and sialylation of antibodies.

EMBODIMENT 6: The method of any one of EMBODIMENTS 2 to 5, wherein the antibody samples comprise monoclonal antibody samples.

EMBODIMENT 7: The method of any one of EMBODIMENTS 1 to 6, wherein training the machine learning model comprises selecting the set of predictors from a plurality of combinations of the related biophysical attributes of therapeutic proteins.

EMBODIMENT 8: The method of EMBODIMENT 7, wherein selecting the set of predictors comprises repeated random subsampling validation.

EMBODIMENT 9: The method of EMBODIMENTS 7 or 8, wherein selecting the set of predictors comprises cross-validation using a pre-defined split of the first input data.

EMBODIMENT 10: The method of any one of EMBODIMENTS 1 to 9, wherein training the machine learning model comprises selecting the machine learning model if the machine learning model is determined to have a model performance that meets a predefined threshold using the first input data and the set of predictors.

EMBODIMENT 11: The method of any one of EMBODIMENTS 1 to 11, further comprising selecting a therapeutic candidate from the second set of therapeutic protein samples based on the predicted functional response.

EMBODIMENT 12: The method of any one of EMBODIMENT 11, further comprising validating a therapeutic efficacy of the therapeutic candidate.

EMBODIMENT 13: The method of any one of EMBODIMENTS 11 or 12, further comprising developing a therapeutic compositing comprising the therapeutic candidate.

EMBODIMENT 14: The method of any one of EMBODIMENTS 1 to 13, wherein the machine learning model is a model based on partial least square, random forest, support vector machine, Naive Bayes, KNN, Generalized additive model, logistic regression, gradient boosting, or lasso.

EMBODIMENT 15: The method of any one of EMBODIMENTS 1 to 14, wherein the machine learning model is a model based on partial least square, random forest, or support vector machine.

EMBODIMENT 16: A system comprising: a data source for obtaining one or more datasets, wherein the one or more datasets comprise: a) first input data related to a set of predictors and corresponding measured functional response associated with the set of predictors obtained from a first set of therapeutic protein samples and b) second input data related to the set of predictors and a second set of therapeutic protein samples for prediction of a functional response, wherein the set of predictors were selected as a combination of related biophysical attributes of therapeutic proteins based on a pre-determined criterion; a computing device communicatively connected to the data source and configured to receive the dataset, the computing device comprising a non-transitory computer readable storage medium containing instructions which, when executed on one or more data processors, cause the one or more data processors to perform a method, the method comprising: training a machine learning model with the first input data; using the machine learning model and the set of predictors to predict a functional response of the second set of therapeutic protein samples based on the second input data; and returning an output comprising the predicted functional response.

EMBODIMENT 17: The system of EMBODIMENT 16, wherein the therapeutic protein samples are antibody samples, the functional response is antibody-dependent cell-mediated cytotoxicity (ADCC) response, complement-dependent cytotoxicity (CDC) response, Fc gamma receptors (FcyR) binding or complement Clq binding, and the related biophysical attributes of therapeutic proteins comprise a degree of afucosylation and one or more glycosylation attributes of antibodies.

EMBODIMENT 18: The system of EMBODIMENTS 16 or 17, wherein training the machine learning model comprises selecting the set of predictors from a plurality of combinations of the related biophysical attributes of therapeutic proteins.

EMBODIMENT 19: The system of EMBODIMENT 18, wherein selecting the set of predictors comprises repeated random subsampling validation.

EMBODIMENT 20: The system of EMBODIMENTS 18 or 19, wherein selecting the set of predictors comprises cross-validation using a pre-defined split of the first input data.

EMBODIMENT 21: The system of any one of EMBODIMENTS 16 to 20, wherein training the machine learning model comprises selecting the machine learning model if the machine learning model is determined to have a model performance that meets a predefined threshold using the first input data and the set of predictors.

EMBODIMENT 22: The system of any one of EMBODIMENTS 16 to 21, wherein the first set of therapeutic protein samples or the second set of therapeutic protein samples comprise antibody samples.

EMBODIMENT 23: The system of any one of EMBODIMENTS 16 to 22, wherein the method further comprises selecting a therapeutic candidate from the second set of therapeutic protein samples based on the predicted functional response.

EMBODIMENT 24: The system of any one of EMBODIMENTS 16 to 23, wherein the machine learning model is a model based on partial least square, random forest, or support vector machine.

EMBODIMENT 25: A computer-program product tangibly embodied in a non-transitory machine-readable storage medium, including instructions configured to cause one or more data processors to perform a method for selecting a cell of interest based on a single cell dataset, the method comprising: receiving input data comprising: a) first input data related to a set of predictors and corresponding measured functional response associated with the set of predictors obtained from a first set of therapeutic protein samples and b) second input data related to the set of predictors and a second set of therapeutic protein samples for prediction of a functional response, wherein the set of predictors were selected as a combination of related biophysical attributes of therapeutic proteins based on a pre-determined criterion; training a machine learning model with the first input data; using the machine learning model and the set of predictors to predict a functional response of the second set of therapeutic protein samples based on the second input data; and returning an output comprising the predicted functional response.

EMBODIMENT 26: The computer-program product of EMBODIMENT 25, wherein therapeutic protein samples are antibody samples, the functional response is antibody-dependent cell-mediated cytotoxicity (ADCC) response, complement-dependent cytotoxicity (CDC) response, Fc gamma receptors (FcyR) binding or complement Clq binding, and the related biophysical attributes of therapeutic proteins comprise a degree of afucosylation and one or more glycosylation attributes of antibodies.

EMBODIMENT 27: The computer-program product of EMBODIMENTS 25 or 26, wherein training the machine learning model comprises selecting the set of predictors from a plurality of combinations of the related biophysical attributes of therapeutic proteins.

EMBODIMENT 28: The computer-program product of EMBODIMENT V, wherein selecting the set of predictors comprises repeated random subsampling validation.

EMBODIMENT 29: The computer-program product of EMBODIMENTS 27 or 28, wherein selecting the set of predictors comprises cross-validation using a pre-defined split of the first input data.

EMBODIMENT 30: The computer-program product of any one of EMBODIMENTS 25 to 29, wherein training the machine learning model comprises selecting the machine learning model if the machine learning model is determined to have a model performance that meets a predefined threshold using the first input data and the set of predictors.

EMBODIMENT 31: The computer-program product of any one of EMBODIMENTS 25 to 30, wherein the first set of therapeutic protein samples or the second set of therapeutic protein samples comprise antibody samples.

EMBODIMENT 32: The computer-program product of any one of EMBODIMENTS 25 to 31, wherein the method further comprises selecting a therapeutic candidate from the second set of therapeutic protein samples based on the predicted functional response.

EMBODIMENT 33: The computer-program product of any one of EMBODIMENTS 25 to 32, wherein the machine learning model is a model based on partial least square, random forest, or support vector machine.

1. A method comprising:
receiving input data comprising:
a) first input data related to a set of predictors and corresponding measured functional response associated with the set of predictors obtained from a first set of therapeutic protein samples and
b) second input data related to the set of predictors and a second set of therapeutic protein samples for prediction of a functional response,
wherein the set of predictors were selected as a combination of related biophysical attributes of therapeutic proteins based on a pre-determined criterion;
training a machine learning model with the first input data;
using the machine learning model and the set of predictors to predict a functional response of the second set of therapeutic protein samples based on the second input data; and
returning an output comprising the predicted functional response.

2. The method of claim 1, wherein the therapeutic protein samples are antibody samples, the functional response is antibody-dependent cell-mediated cytotoxicity (ADCC) response, complement-dependent cytotoxicity (CDC) response, Fc gamma receptors (FcyR) binding or complement Clq binding, and the related biophysical attributes of therapeutic proteins comprise a degree of afucosylation and one or more additional glycosylation attributes of antibodies.

3. The method of claim 2, wherein the one or more additional glycosylation attributes of antibodies comprise galactosylation, sialylation, glycan chain length, glycan building block type, and forms of antibodies missing N-glycan chains, or any combination thereof.

4. The method of claim 2, wherein the one or more additional glycosylation attributes of antibodies comprise two glycosylation attributes of antibodies.

5. The method of claim 2, wherein the one or more additional glycosylation attributes of antibodies comprise galactosylation and sialylation of antibodies.

6. The method of claim 2, wherein the antibody samples comprise monoclonal antibody samples.

7. The method of claim 1, wherein training the machine learning model comprises selecting the set of predictors from a plurality of combinations of the related biophysical attributes of therapeutic proteins.

8. The method of claim 7, wherein selecting the set of predictors comprises repeated random subsampling validation.

9. The method of claim 7, wherein selecting the set of predictors comprises cross-validation using a pre-defined split of the first input data.

10. The method of claim 1, wherein training the machine learning model comprises selecting the machine learning model if the machine learning model is determined to have a model performance that meets a predefined threshold using the first input data and the set of predictors.

11. The method of claim 1, further comprising selecting a therapeutic candidate from the second set of therapeutic protein samples based on the predicted functional response.

12. The method of claim 11, further comprising validating a therapeutic efficacy of the therapeutic candidate.

13. The method of claim 11, further comprising developing a therapeutic compositing comprising the therapeutic candidate.

14. The method of claim 1, wherein the machine learning model is a model based on partial least square, random forest, support vector machine, Naive Bayes, KNN, Generalized additive model, logistic regression, gradient boosting, or lasso.

15. The method of claim 1, wherein the machine learning model is a model based on partial least square, random forest, or support vector machine.

16. A system comprising:
a data source for obtaining one or more datasets, wherein the one or more datasets comprise:
a) first input data related to a set of predictors and corresponding measured functional response associated with the set of predictors obtained from a first set of therapeutic protein samples and
b) second input data related to the set of predictors and a second set of therapeutic protein samples for prediction of a functional response,
wherein the set of predictors were selected as a combination of related biophysical attributes of therapeutic proteins based on a pre-determined criterion;
a computing device communicatively connected to the data source and configured to receive the dataset, the computing device comprising a non-transitory computer readable storage medium containing instructions which, when executed on one or more data processors, cause the one or more data processors to perform a method, the method comprising:
training a machine learning model with the first input data;
using the machine learning model and the set of predictors to predict a functional response of the second set of therapeutic protein samples based on the second input data; and
returning an output comprising the predicted functional response.

17. The system of claim 16, wherein the therapeutic protein samples are antibody samples, the functional response is antibody-dependent cell-mediated cytotoxicity (ADCC) response, complement-dependent cytotoxicity (CDC) response, Fc gamma receptors (FcyR) binding or complement Clq binding, and the related biophysical attributes of therapeutic proteins comprise a degree of afucosylation and one or more glycosylation attributes of antibodies.

18. The system of claim 16, wherein training the machine teaming model comprises selecting the set of predictors from a plurality of combinations of the related biophysical attributes of therapeutic proteins.

19. The system of claim 18, wherein selecting the set of predictors comprises repeated random subsampling validation.

20. The system of claim 18, wherein selecting the set of predictors comprises cross-validation using a pre-defined split of the first input data.

21. The system of claim 16, wherein training the machine learning model comprises selecting the machine learning model if the machine learning model is determined to have a model performance that meets a predefined threshold using the first input data and the set of predictors.

22. The system of claim 16, wherein the first set of therapeutic protein samples or the second set of therapeutic protein samples comprise antibody samples.

23. The system of claim 16, wherein the method further comprises selecting a therapeutic candidate from the second set of therapeutic protein samples based on the predicted functional response.

24. The system of claim 16, wherein the machine learning model is a model based on partial least square, random forest, or support vector machine.

25. A computer-program product tangibly embodied in a non-transitory machine-readable storage medium, including instructions configured to cause one or more data processors to perform a method for selecting a cell of interest based on a single cell dataset, the method comprising:
receiving input data comprising:
a) first input data related to a set of predictors and corresponding measured functional response associated with the set of predictors obtained from a first set of therapeutic protein samples and b) second input data related to the set of predictors and a second set of therapeutic protein samples for prediction of a functional response,
wherein the set of predictors were selected as a combination of related biophysical attributes of therapeutic proteins based on a pre-determined criterion;
training a machine learning model with the first input data;
using the machine learning model and the set of predictors to predict a functional response of the second set of therapeutic protein samples based on the second input data; and
returning an output comprising the predicted functional response.

26. The computer-program product of claim 25, wherein therapeutic protein samples are antibody samples, the functional response is antibody-dependent cell-mediated cytotoxicity (ADCC) response, complement-dependent cytotoxicity (CDC) response, Fc gamma receptors (FcyR) binding or complement Clq binding, and the related biophysical attributes of therapeutic proteins comprise a degree of afucosylation and one or more glycosylation attributes of antibodies.

27. The computer-program product of claim 25, wherein training the machine learning model comprises selecting the set of predictors from a plurality of combinations of the related biophysical attributes of therapeutic proteins.

28. The computer-program product of claim 27, wherein selecting the set of predictors comprises repeated random subsampling validation.

29. The computer-program product of claim 27, wherein selecting the set of predictors comprises cross-validation using a pre-defined split of the first input data.

30. The computer-program product of claim 25, wherein training the machine learning model comprises selecting the machine learning model if the machine learning model is determined to have a model performance that meets a predefined threshold using the first input data and the set of predictors.

31. The computer-program product of claim 25, wherein the first set of therapeutic protein samples or the second set of therapeutic protein samples comprise antibody samples.

32. The computer-program product of claim 25, wherein the method further comprises selecting a therapeutic candidate from the second set of therapeutic protein samples based on the predicted functional response.

33. The computer-program product of claim 25, wherein the machine learning model is a model based on partial least square, random forest, or support vector machine.
